(19) United States
(12) Patent Application Publication
(10) Pub. No.: US 2022/0218725 A1
(43) Pub. Date: Jul. 14, 2022

(54) METHODS AND MATERIALS FOR TREATING CANCER

(71) Applicant: Mayo Foundation for Medical Education and Research, Rochester, MN (US)

(72) Inventors: Thomas E. Witzig, Rochester, MN (US); Xiaosheng Wu, Rochester, MN (US); Jithma P. Abeykoon, Rochester, MN (US)

(21) Appl. No.: 17/610,487

(22) PCT Filed: May 18, 2020
(86) PCT No.: PCT/US2020/033413

§ 371 Date: Nov. 11, 2021

Related U.S. Application Data
(60) Provisional application No. 62/848,948, filed on May 16, 2019.

(51) Int. Cl. A61K 31/618; A61K 31/497; A61K 31/366; A61K 31/4196; A61K 31/506; A61K 31/4412; A61K 31/4439; A61K 31/352; A61P 31/14

(52) U.S. Cl. CPC A61K 31/618; A61K 31/497; A61K 31/366; A61K 31/4196; A61P 31/14; A61K 31/4412; A61K 31/4439; A61K 31/352; A61K 31/506

(57) ABSTRACT

This document provides methods and materials involved in treating cancer. For example, this document provides methods and materials for using one or more inhibitors of a chromosomal maintenance 1 (CRM1) polypeptide in combination with one or more salicylates to treat cancer in a mammal (e.g., a human).

### CROSS-REFERENCE TO RELATED APPLICATIONS

This application claims the benefit of U.S. Patent Application Ser. No. 62/848,948, filed on May 16, 2019. The disclosure of the prior application is considered part of (and is incorporated by reference in) the disclosure of this application.

### BACKGROUND

### 1. Technical Field

This document relates to methods and materials involved in treating cancer. For example, this document provides methods and materials for using one or more inhibitors of a chromosomal maintenance 1 (CRM1) polypeptide in combination with one or more salicylates to treat cancer in a mammal (e.g., a human).

### 2. Background Information

Cancer cells can use nucleo-cytoplasmic trafficking of macromolecules to sustain proliferation and survival. Chromosome region maintenance 1 (CRM1), also known as Exportin-1 (XPO1), is a transport receptor that mediates nuclear efflux of proteins having a leucine-rich nuclear export sequence (NES). Tumors with increased expression of CRM1 polypeptides can export tumor suppressive polypeptides out of the nucleus to the cytoplasm and thus can disable the cells' tumor suppressor activities. Therefore, drugs that target CRM1 polypeptides have become attractive as a therapeutic option for cancer, and selinexor (KPT-330) has been used in phase 1, 2 and 3 clinical trials in patients with multiple myeloma (MM) and non-Hodgkin lymphoma (NHL). Although selinexor has shown promising antitumor effects at high concentrations, the drug related adverse effects hinder its potential in becoming a potent anticancer drug in patients with hematologic malignancies.

### SUMMARY

This document provides methods and materials involved in treating cancer. For example, one or more inhibitors of a CRM1 polypeptide in combination with one or more salicylates can be used as described herein to treat cancer in a mammal (e.g., a human). In some cases, an inhibitor of a CRM1 polypeptide can be administered to a mammal (e.g., a human) having a cancer in a low concentration (e.g., a low dose). A low concentration of an inhibitor of a CRM1 polypeptide (e.g., selinexor) refers to any concentration of an inhibitor of a CRM1 polypeptide that is less than about 160 mg/week (e.g., about 80 mg twice weekly), less than about 45 mg/m² body area twice weekly, or less than about 1.25 μM plasma concentration.

As demonstrated herein, antitumor effects of low concentrations of an inhibitor of a CRM1 polypeptide (e.g., selinexor and leptomycin B) can be enhanced when the inhibitor of a CRM1 polypeptide is administered in combination with one or more salicylates (e.g., aspirin, choline salicylate, and/or sodium salicylate). Having the ability to treat a mammal (e.g., a human) having cancer with one or more inhibitors of a CRM1 polypeptide in combination with one or more salicylates provides an opportunity for the mammal to benefit from the antitumor effects of the inhibitor(s) of a CRM1 polypeptide without experiencing drug related adverse effects. For example, using one or more inhibitors of a CRM1 polypeptide in combination with one or more salicylates can allow the inhibitor(s) of a CRM1 polypeptide to be administered to a mammal (e.g., a human) having cancer at lower concentrations, making inhibitor(s) of a CRM1 polypeptide more tolerable and appealing option for treating a mammal having cancer.

In general, one aspect of this document features methods for treating a mammal having cancer. The methods can include, or consist essentially of, administering (a) an inhibitor of a chromosomal maintenance 1 (CRM1) polypeptide and (b) a salicylate to the mammal to reduce the number of cancer cells in the mammal. The mammal can be a human.

The cancer can be a hematologic cancer. The cancer can be diffuse large B-cell lymphoma (DLBCL), a T-cell lymphoma (TCL), a mantle cell lymphoma (MCL), a non-Hodgkin lymphoma (NHL), multiple myeloma (MM), Hodgkin lymphoma, small lymphocytic lymphoma, lymphoplasmacytic lymphoma, chronic lymphocytic leukemia, chronic lymphocytic leukemia, acute myelogenous leukemia, chronic myelogenous leukemia, myeloproliferative syndromes, or myelodysplastic syndromes. The inhibitor of a CRM1 polypeptide can be selinexor, leptomycin B, KPT-185, KPT-276, eltanexor, piperlongumine, verdinexor, valtrate, or ratjadone C. The inhibitor of a CRM1 polypeptide can result in a plasma concentration within the mammal of from about 0.01 nM to about 1.25 μM (e.g., from about 0.25 μM to about 1.24 μM). The salicylate can be aspirin, choline salicylate, sodium salicylate, acetyl salicylate, or choline magnesium trisalicylate. The salicylate can result in a plasma concentration within the mammal of from about 0.1 μM to about 10 mM.

In another aspect, this document features methods for treating a mammal having cancer. The methods can include, or consist essentially of, administering (a) an inhibitor of a CRM1 polypeptide and (b) a salicylate to the mammal to arrest the cell cycle of a cancer cell in the mammal. The cell cycle can be arrested at a S phase. The mammal can be a human. The cancer can be a hematologic cancer. The cancer can be diffuse large B-cell lymphoma (DLBCL), a T-cell lymphoma (TCL), a mantle cell lymphoma (MCL), a non-Hodgkin lymphoma (NHL), multiple myeloma (MM), Hodgkin lymphoma, small lymphocytic lymphoma, lymphoplasmacytic lymphoma, chronic lymphocytic leukemia, chronic lymphocytic leukemia, acute myelogenous leukemia, chronic myelogenous leukemia, myeloproliferative syndromes, or myelodysplastic syndromes. The inhibitor of a CRM1 polypeptide can be selinexor, leptomycin B, KPT-185, KPT-276, eltanexor, piperlongumine, verdinexor, valtrate, or ratjadone C. The inhibitor of a CRM1 polypeptide can result in a plasma concentration within the mammal of from about 0.01 nM to about 1.25 μM (e.g., from about 0.25 μM to about 1.24 μM). The salicylate can be aspirin, choline salicylate, sodium salicylate, acetyl salicylate, or choline magnesium trisalicylate. The salicylate can result in a plasma concentration within the mammal of from about 0.1 μM to about 10 mM.

In another aspect, this document features methods for treating a mammal having a viral infection. The methods can include, or consist essentially of, administering (a) an inhibitor of a chromosomal maintenance 1 (CRM1) polypeptide and (b) a salicylate to the mammal to reduce the number of viral particles in the mammal. The mammal can be a human. The viral infection can be caused by a coronavirus. The coronavirus can be a betacoronavirus. The virus can be SARS-CoV-2. The inhibitor of a CRM1 polypeptide can be selinexor, leptomycin B, KPT-185, KPT-276, eltanexor, piperlongumine, verdinexor, valtrate, or ratjadone C. The inhibitor of a CRM1 polypeptide can result in a plasma concentration within the mammal of from about 0.01 nM to about 1.25 μM. The salicylate can be aspirin, choline salicylate, sodium salicylate, acetyl salicylate, or choline magnesium trisalicylate.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. Cell viability of diffuse large B-cell lymphoma cells (Ly-1 cell line) following combination drug treatment with selinexor (KPT) at 2.0 μM and choline salicylate (CS) at 3 mM. Similar results were seen when selinexor was used at 0.1 μM to 2.0 μM with CS at 1 mM, 2 mM, 3 mM, 4 mM, and 5 mM.

FIG. 2. Cell viability of mantle cell lymphoma cells (Jeko-1 cell line) following combination drug treatment with selinexor (KPT) at 0.5 μM and CS at 3 mM.

FIG. 3A. Cell proliferation of mantle cell lymphoma cells (Jeko-1 cell line) following combination drug treatment with selinexor (KPT) at 0.5 μM and CS at 3 mM.

FIG. 3B. Cell cycle phase of mantle cell lymphoma cells (Jeko-1 cell line) following combination drug treatment with selinexor (KPT) at 0.5 μM and CS at 3 mM.

FIG. 4. Cell viability of diffuse large B-cell lymphoma cells (Ly-1 cell line) treated with selinexor (KPT) in combination with CS or aspirin (Asp).

FIG. 5. Cell viability of diffuse large B-cell lymphoma cells (Ly-1 cell line) treated with selinexor (KPT-330 or K) in combination with CS or sodium salicylate (Na-Sal).

FIG. 6. IC₅₀ of mantle cell lymphoma cells (Jeko-1 cell line) following treatment with selinexor (KPT-330) alone or combination drug treatment with selinexor and CS.

FIG. 7. IC₅₀ of diffuse large B-cell lymphoma cells (Ly-1 cell line) following treatment with selinexor (KPT-330) alone or combination drug treatment with selinexor and CS.

FIG. 8. CRM1 expression in cells treated with selinexor (KPT-330) or KPT-185 in combination with CS.

FIG. 9A. Cell viability of mantle cell lymphoma cells (Jeko-1 cell line) and of diffuse large B-cell lymphoma cells (Ly-1 cell line) treated with selinexor (KPT-330) in combination with ketorolac.

FIG. 9B. Cell viability of mantle cell lymphoma cells (Jeko-1 cell line) treated with selinexor in combination with ketorolac in high concentrations.

FIG. 10. Cell viability of diffuse large B-cell lymphoma cells (Ly-1 cell line) treated with selinexor (KPT-330) in combination with ibuprofen (ibu).

FIG. 11. Cell viability of mantle cell lymphoma cells (Jeko-1 cell line) and of diffuse large B-cell lymphoma cells (Ly-1 cell line) treated with aspirin in combination with gemcitabine.

FIG. 12. Cell viability of diffuse large B-cell lymphoma cells (Ly-1 cell line) treated with selinexor (KPT) in combination with CS or bortezomib (bortz).

FIG. 13. Cell viability of diffuse large B-cell lymphoma cells (Ly-1 cell line) treated with different concentrations of selinexor (KPT) in combination with different concentrations of CS.

FIG. 14. Cell viability of diffuse large B-cell lymphoma cells (Ly-1 cell line) treated with selinexor (KPT) at 1.0 μM or 2.0 μM in combination with CS at 3 mM. Similar results were seen when selinexor was used at 0.1 μM to 2.0 μM with CS at 1 mM, 2 mM, 3 mM, 4 mM, and 5 mM.

FIG. 15. Cell viability of diffuse large B-cell lymphoma cells (Ly-3 cell line) treated with selinexor (KPT) at 0.25 μM, 0.5 μM, or 1.0 μM in combination with CS at 1 mM or 2 mM.

FIG. 16. Cell viability of mantle cell lymphoma cells (Jeko-1 cell line) treated with selinexor (KPT-330) at 0.25 μM, 0.5 μM, or 1.0 μM in combination with CS at 1 mM, 2 mM, 3 mL, or 4 mM.

FIG. 17. Tumor size within mice treated with selinexor (KPT-330) alone or in combination with CS.

FIG. 18. Tumors removed from sacrificed mice treated with selinexor (KPT-330) alone or in combination with CS.

FIG. 19. KPT-330+CS potentiates the antitumor effect of CRM1 inhibitors ex vivo. (a-d): Cell viability analysis using Annexin/PI analysis on JeKo-1 cell treated with various salicylates (AS: 2.5 mM, NaS: 3 mM, CS: 3 mM) and CRM1 inhibitors (LMB: 2 ηM, KPT-185: 0.2 μM, KPT-330: 0.5 μM) in combination or as single agents. The combination of salicylates with CRM1 inhibitors significantly enhanced antitumor activity as compared to either agent alone. (e-f): IC₅₀ was calculated for JeKo-1 (e) and OCI-Ly1 cell lines (f). The IC₅₀ decreased from 1.3 μM to 0.3 μM on JeKo-1 cells, and from 1.8 μM to 0.4 μM on OCI-Ly1 cells when treated with CS 3 mM and KPT-330 at the indicated concentrations. (g) Cell viability analysis using Annexin V/PI analysis on cell lines from different hematologic malignancies and solid tumors treated with KPT-330 (from 0.1p M to 0.5 μM) and CS (from 1 to 3 mM). Results were normalized by the respective controls. The lowest concentrations of both KPT-330 and CS that induced synergy were selected and maintained constant within the treatment conditions for each line. MCL: mantle cell lymphoma; DLBCL: diffuse large B-cell lymphoma; MM: multiple myeloma; TCL: T-cell lymphoma; ALL: acute lymphoblastic leukemia; WM: Waldenstrom macroglobulinemia; AS: acetyl salicylate; CS: choline salicylate; NaS: sodium salicylate; Con: control; LMB: Leptomycin B; Pancreas: pancreatic adenocarcinoma; NSCLC: Non-small cell lung cancer; SCLC: small-cell lung cancer. p<0.05-0.005; **: p<0.005-0.0005; ***: p<0.0005. Paired Student's t-test was used to compare all continuous variables. A p-value of <0.05 was considered statistically significant.

FIG. 20. KPT-330+CS shows potent antitumor effect without substantial in vivo organ toxicity. Tumor volume curves (a) and extracted tumor images (b) of NSG mice transplanted subcutaneously with JeKo-1 cells and treated with vehicle, KPT-330, CS, or KPT-330+CS. Tumor volumes were measured daily for 26 days. (c): Histopathological assessment of organs from non-tumor bearing mice treated with KPT-330+CS or vehicle for 26 days. No significant toxicities were seen in the treatment group compared to controls; grade I renal tubular hyperplasia (black arrowhead) was seen in 4/5 mice as compared to 1/5 mice in the treatment and control groups, respectively. ***: p<0.0005. Paired Student's t-test was used to compare all continuous variables. A p-value of <0.05 was considered statistically significant.

FIG. 21. KPT-330+CS is a better inhibitor of nuclear export and decreases CRM1 expression. (a): Immunofluorescence microscopic images showing the subcellular localization of GFP and endogenous CRM1 in U2OS cells transfected with nuclear export reporter construct (NLS-GFP-NES), and treated with the indicated conditions for 24 hours. (b): Quantitation of nuclear export efficiency. GFP-transfected cells were evaluated and scored for percentage with complete nuclear localization of GFP signal. (c): Western blotting images showing the expression of transfected CRM1-YFP and the endogenous CRM1 in U2OS, HeLa, and HEK293 cells treated with the indicated conditions for 24 hours. *: p=0.02. Paired Student's t-test was used to compare all continuous variables. A p-value of <0.05 was considered statistically significant.

FIG. 22. KPT-330+CS uniquely inhibits proteins in key cellular pathways. (a-e): Volcano plots showing protein changes associated with each treatment condition; (a) KPT-330+CS vs. Control; (b) KPT-330 vs. Control; (c) CS vs. Control; (d) CS vs. KPT-330; (e) KPT-330+CS vs. KPT-330. Significantly differentially expressed proteins (absolute log 2 fold change >=2 and FDR<=0.05) are highlighted. Key proteins in cell cycle, nucleotide synthesis, and DNA damage repair pathways are tagged where detected. (j): Heat map showing the downregulation of selected proteins involved in DNA damage repair, cell cycle progression, DNA synthesis, and nuclear molecular export. Each condition was performed in biological triplicates of the JeKo-1 cell line. (f-h): Immunoblotting images validating the findings of proteomic studies. The selected proteins that are critical in DNA damage repair, DNA synthesis, cell cycle and nucleocytoplasmic molecular transport were uniquely decreased starting at 24 hours following KPT-330+CS treatment in JeKo-1 cells. (i): Heat map showing significantly affected pathways by KPT-330+CS. Pathways related to cell cycle, DNA damage repair, and nucleotide synthesis were significantly downregulated, while pathways associated with apoptosis were upregulated when cells were treated with KPT-330+CS. An ANOVA test was used to detect the differentially expressed protein groups between pairs of experimental groups. Differential expression p-values were FDR corrected using Benjamini-Hochberg procedure. A total of five group comparisons were performed: KPT-330 vs. control, CS vs. control, KPT-330+CS vs. control, KPT-330+CS vs. KPT-330, and CS vs. KPT-330. For each comparison, protein groups with an FDR≤0.05 and an absolute log 2 (fold change)≥2.0 were considered as significantly differentially expressed.

FIG. 23. KPT-330+CS induces cell death through caspase activation. Annexin V/PI assay showing cell viability in JeKo-1 cells treated for 48 hours with KPT-330 (0.5 μM) and CS (3 mM) as single agents and in combination in the presence or absence of a pan-caspase inhibitor (Q-VD-OPh). CS: choline salicylate; ***: p<0.0001. Paired Student's t-test was used to compare all continuous variables. A p-value of <0.05 was considered statistically significant.

FIG. 24. The effect on critical proteins by KPT-330+CS is not due to caspase activation. JeKo-1 cells were treated with KPT-330+CS in the presence or absence of Q-VD-OPh, a pan-caspase inhibitor. Protein expression was assessed through immunoblotting and was compared to controls. The decreased protein expression with KPT-330+CS was not affected by the pan-caspase inhibitor, supporting the fact that the effect of KPT-330+CS on these vital proteins is due to the drug combination and not merely due to caspase-induced programmed cell death. TYMS: thymidylate synthase

FIG. 25. NFkB-mediated cellular signaling is not affected by treatment with KPT-330 or CS as single agent or in combination. (a-c) Gene set enrichment of NFkB genes in protein differential expression derived by comparing single-agent treatments (KPT-330 or CS) and in combination (KPT-330+CS) with controls. (d-f) A similar analysis using gene expression derived from the same comparisons. No statistically significant enrichment of NFkB genes was detected with K+CS treatment at the proteomic or gene expression level. These data suggest that the NFkB pathway is not a significant contributing factor for the cytotoxicity of KPT-330+CS treatment.

FIG. 26. KPT-330+CS arrests cells in S-phase and inhibits DNA damage repair. (a): Cell cycle profile of CS, KPT-330, and KPT-330+CS treated unsynchronized JeKo-1 cells. The KPT-330+CS treatment arrests cells at S-phase (filled arrow) and induces cell death (hollow arrow). (b): Cell cycle profiles of KPT-330+CS treated JeKo-1 cells assessed at different time points from release after G1-phase synchronization. Progression of S-phase arrest with time was observed upon KPT-330+CS (filled arrow) while increasing cell death (hollow arrow). (c): γH2AX foci formed in JeKo-1 cells following KPT-330+CS treatment. (d): Immunoblotting confirmed the appearance of γ-H2AX starting at 24 hours in JeKo-1 cells with concomitant decrease of Rad51 expression after KPT-330+CS treatment. (e): Comet assay indicated DNA damage (comet tail) in JeKo-1 cells treated with KPT-330+CS. The KPT-330+CS treatment uniquely increased the prevalence of JeKo-1 cells with characteristic comet tails, indicating the presence of DNA damage. (f): PARP inhibitors potentiate the antitumor effect of the combination drug treatment. JeKo-1 cells were treated with KPT-330 (0.5 μM) and CS (3 mM) as single agent or in combination in the presence or absence of olaparib (10 μM). Combining KPT+CS with olaparib induced significantly better cell killing as assessed by AnnexinV/PI assay following 48 h of incubation (p=0.00016). **: p=0.0016. Paired Student's t-test was used to compare all continuous variables. A p-value of <0.05 was considered statistically significant.

FIG. 27. Expression of selected proteins in different phases of cell cycle. Immunoblot showing expression of selected proteins in cell cycle synchronized JeKo-1 cells. Untreated JeKo-1 cells were synchronized at G1-phase by thymidine double block. After releasing the cells (t=0 hours), the expression of proteins were assessed. We focused on proteins identified (FIG. 22f) to be affected by KPT-330+CS and the time period between 8 and 16 hours (FIG. 26b) where the G2/M-phase of the cell cycle becomes prominent. Indeed, in accordance with those results, the immunoblot shown here depicts increased expression of the G2/M-phase specific proteins; PLK1, Bub1b, and Aurora A, at approximately the same time period where G2/M-phase become prominent. The expression of PLK1, Bub1b, and Aurora A decreased after 16 h as cells exited the G2/M-phase. Conversely, the expression of Rad51 and TYMS were not cell-cycle specific and did not fluctuate following release. TYMS: thymidylate synthase

FIG. 28. KPT-330+CS causes decreased expression of Rad51 protein and increased expression of γ-H2AX in a primary patient sample of marginal zone lymphoma. Immunoblot showing expression of Rad51 and γ-H2AX in a primary patient sample. Mononuclear cells were obtained from spleen tissue of a patient with relapsed marginal zone lymphoma. Protein expression was assessed by immunoblot following treatment with KPT-330+CS or DMSO control for 24 hours. A substantial decrease in Rad51 protein with simultaneous appearance of γ-H2AX was observed upon KPT-330+CS treatment. MZL: marginal zone lymphoma; CS: choline salicylate

FIG. 29. KPT-330+CS causes decreased expression of TYMS in primary patient samples. Immunoblot showing expression of TYMS in primary patient samples. Mononuclear cells were obtained from spleen tissue of a patient with relapsed marginal zone lymphoma, and from a lymph node of a patient with relapsed DLBCL. Protein expression was assessed following treatment with KPT-330+CS or DMSO control for 24 hours. A substantial decrease in TYMS was observed upon KPT-330+CS treatment. MZL: marginal zone lymphoma; CS: choline salicylate; TYMS: thymidylate synthase; DLBCL: diffuse large B-cell lymphoma.

FIG. 30. Thymidine partially reverses the effect of KPT-330+CS on lymphoma cells. Viability assay showing the effect of KPT-330, CS and KPT-330+CS with or without exogenous thymidine. JeKo-1 cells were cultured in thymidine-free media and treated with 0.5 μM KPT-330 and 3 mM CS in combination or as single agent with or without 10 μM of thymidine. Viability was assessed by Annexin V/PI assay. Statistically significant recovery (p=0.005) of cell viability was observed. CS: choline salicylate; ***: p=0.005; Paired Student's t-test was used to compare all continuous variables. A p-value of <0.05 was considered statistically significant.

FIG. 31. Gene expression changes in the JeKo-1 cell line with KPT-330 or CS treatment as a single agent or in combination. JeKo-1 cells were treated for 24-hours with DMSO (Control), KPT-330 alone, CS alone, or KPT-330+CS. Treated cells were subjected to gene expression analysis using mRNA sequencing (RNA-Seq), with significantly differentially expressed proteins detected (absolute log 2 fold change >=2 and adjusted p-value<=0.05). (a-d) Volcano plots show the profile of mRNA changes associated with each treatment condition when compared to control. Numbers in boxes summarize the total number of up regulated (numerator) and down regulated (denominator) genes. (e) We identified a total of 227 genes that were significantly differentially expressed (absolute log 2 fold change >=2 and adjusted p-value<=0.05) by the KPT-330+CS treatment and the heat map shows the clustering of gene expression and protein expression changes. Both protein and gene expression analyses concur with decreased expression of proteins related to cell cycle, DNA damage repair and nucleotide synthesis, while upregulation of proteins related to apoptosis and cell death.

FIG. 32. KPT-330+CS imposes significant antitumor effect on primary patient samples. (a): Viability assay showing the effect of KPT-330, CS and KPT-330+CS on mononuclear cells obtained from primary patient tissue samples. KPT-330 concentration ranging from 0.1 μM to 0.5 μM, and CS concentration ranging from 1 mM to 3 mM as single agents or in combination were used and treated for 48 hours. The lowest concentrations of KPT-330 and CS which gave best synergy were selected and kept constant across conditions. (b): Viability assay showing the effect of KPT-330, CS and KPT-330+CS on mononuclear cells obtained from peripheral blood of healthy donors without a flow cytometry proven diagnosis of malignancy, and tissue biopsies from the indicated tissue types from patients without a diagnosis of cancer per histopathologic review. Cells were treated with 0.5 μM KPT-330 and 3 mM CS as single agent or in combination. (c): Patient tumor samples derived from PDX (5 ovarian cancers and 2 gliomas) were treated ex vivo with KPT-330 and CS as single agents or in combination. Ovarian tumor cells were treated with 0.6 μM KPT-330 and 0.6 mM CS as single agent or in combination. Glioma cells were treated with 0.1 μM KPT-330 and 0.3 mM CS as single agent or in combination. MZL: marginal zone lymphoma; CMML: chronic myelomonocytic leukemia; DLBCL: diffuse large B-cell lymphoma; MCL: mantle cell lymphoma; TCL: T-cell lymphoma; LPL: lymphoplasmacytic lymphoma with IgG monoclonal gammopathy; WM: Waldenstrom macroglobulinemia; CLL: chronic lymphocytic leukemia; BM: bone marrow; PBMC: peripheral blood mononuclear cells; PDX: patient derived xenograft; *: p<0.05-0.005; **: p<0.005-0.0005; ***: p<0.0005. Paired Student's t-test was used to compare all continuous variables. A p-value of <0.05 was considered statistically significant.

FIG. 33. KPT-330+CS induces strong antitumor effect ex vivo on ovarian cancer. Celltiter-Glo® assay showing cell viability following KPT-330 or CS treatment as single agent or in combination. The antitumor effect was assessed ex vivo using tissue derived from PDX. KPT-330 and CS were treated in a concentration dependent manner, and the antitumor effect was assessed by using celltiter-Glo® assay. PDX: patient derived xenografts; CI: combination index which <1 considered as synergistic. Patient IDs: PH013, PH039, PH038, PH080, and PH095.

FIG. 34. SDS-PAGE of Cell Lysates for Proteomics. Sodium dodecyl sulfate-polyacrylamide gel electrophoresis was done in triplicate from each treatment group. Replicates are designated with numbers (1, 2, and 3). Excised bands are designated using short red lines. SDS-PAGE: Sodium dodecyl sulfate-polyacrylamide gel electrophoresis; cont: control; K: KPT-330; CS: choline salicylate; CS+K: choline salicylate+KPT-330; mix: combined control lysate of all treatment groups.

FIG. 35. Representative cytokine profile with selinexor (KPT-330)+CS.

FIG. 36. Levels of cytokines in PMBCs treated with KPT-330 in the presence or absence of choline salicylate (CS).

### DETAILED DESCRIPTION

This document provides methods and materials involved in treating cancer. For example, one or more inhibitors of a CRM1 polypeptide and one or more salicylates (e.g., a composition including one or more inhibitors of a CRM1 polypeptide and one or more salicylates) can be administered to a mammal (e.g., a human) having cancer to treat the mammal. In some cases, one or more inhibitors of a CRM1 polypeptide and one or more salicylates can be administered to a mammal having cancer to reduce the severity of the cancer, to reduce one or more symptoms of the cancer, and/or to reduce the number of cancer cells present within the mammal. For example, one or more inhibitors of a CRM1 polypeptide and one or more salicylates can be administered to a mammal having cancer to reduce one or more symptoms of the cancer by, for example, 10, 20, 30, 40, 50, 60, 70, 80, 90, 95, or more percent. For example, one or more inhibitors of a CRM1 polypeptide and one or more salicylates can be administered to a mammal having cancer to reduce the number of cancer cells present within the mammal by, for example, 10, 20, 30, 40, 50, 60, 70, 80, 90, 95, or more percent.

Alternatively, the methods and materials described herein can be used for treating having, or at risk of developing, a viral infection (e.g., a coronavirus infection) and/or bacterial infection. In some cases, one or more inhibitors of a CRM1 polypeptide and one or more salicylates can be administered to a mammal having, or at risk of developing, a viral infection (e.g., a coronavirus infection) to reduce the number of viral particles within the mammal by, for example, 10, 20, 30, 40, 50, 60, 70, 80, 90, 95, or more percent. In some cases, one or more inhibitors of a CRM1 polypeptide and one or more salicylates can be administered to a mammal having, or at risk of developing, a viral infection (e.g., a coronavirus infection) and/or bacterial infection to reduce one or more symptoms of the viral infection and/or the bacterial infection by, for example, 10, 20, 30, 40, 50, 60, 70, 80, 90, 95, or more percent. In some cases, one or more inhibitors of a CRM1 polypeptide and one or more salicylates can be administered to a mammal having, or at risk of developing, a viral infection (e.g., a coronavirus infection) to reduce viral shedding by the mammal by, for example, 10, 20, 30, 40, 50, 60, 70, 80, 90, 95, or more percent.

Alternatively, the methods and materials described herein can be used for treating having a disease or disorder associated with inflammation (e.g., associated with a pro-inflammatory state). Examples of diseases and disorders associated with inflammation include, without limitation, acute respiratory distress syndrome (ARDS), infections (e.g., viral infections), and autoimmune conditions such as but not limited to rheumatoid arthritis, inflammatory bowel disease, multiple sclerosis, myasthenia gravis and autoimmune cytopenia. In some cases, one or more inhibitors of a CRM1 polypeptide and one or more salicylates can be administered to a mammal having disease or disorder associated with inflammation to reduce the inflammation within the mammal by, for example, 10, 20, 30, 40, 50, 60, 70, 80, 90, 95, or more percent. In some cases, one or more inhibitors of a CRM1 polypeptide and one or more salicylates can be administered to a mammal having disease or disorder associated with inflammation to reduce a level of one or more pro-inflammatory cytokines within the mammal by, for example, 10, 20, 30, 40, 50, 60, 70, 80, 90, 95, or more percent.

In some cases, an inhibitor of a CRM1 polypeptide can be administered to a mammal (e.g., a human) having a cancer in a low concentration (e.g., a low dose). A low concentration of an inhibitor of a CRM1 polypeptide can refer to any concentration of an inhibitor of a CRM1 polypeptide that is less than about 160 mg/week (e.g., about 80 mg twice weekly). A low concentration of an inhibitor of a CRM1 polypeptide (e.g., selinexor) can refer to any concentration of an inhibitor of a CRM1 polypeptide that is less than about 45 mg/m² body area twice weekly. A low concentration of an inhibitor of a CRM1 polypeptide (e.g., selinexor) can refer to any concentration of an inhibitor of a CRM1 polypeptide that is less than about 1.25 μM (e.g., less than about 1.24 μM) plasma concentration. In some cases, a low concentration of an inhibitor of a CRM1 polypeptide (e.g., selinexor) that can be used as described herein can be from about 0.00001 μM (0.01 nM) to about 1 μM plasma concentration (e.g., from about 0.01 nM to about 0.75 μM, from about 0.01 nM to about 0.5 μM, from about 0.01 nM to about 0.25 μM, from about 0.01 nM to about 1 μM, from about 0.01 nM to about 0.5 μM, from about 0.05 nM to about 1 μM, from about 1 nM to about 1 μM, from about 0.1 μM to about 1 μM, from about 1 μM to about 1 μM, from about 0.25 μM to about 1 μM, from about 0.5 μM to about 1 μM, from about 0.75 μM to about 1 μM, from about 0.1 μM to about 0.8 μM, from about 0.25 μM to about 0.75 μM, from about 0.25 μM to about 0.5 μM, from about 0.25 μM to about 0.3 μM, from about 0.3 μM to about 1 μM, from about 0.4 μM to about 1 μM, from about 0.5 μM to about 1 μM, from about 0.7 μM to about 1 μM, or from about 0.4 μM to about 0.8 μM plasma concentration). For example, when an inhibitor of a CRM1 polypeptide is selinexor, a low concentration selinexor can be from about 0.25 μM to about 1.24 μM.

Any appropriate mammal having cancer and/or having, or at risk of developing, a viral infection (e.g., a coronavirus infection) and/or bacterial infection can be treated as described herein. For example, humans and other primates such as monkeys having cancer and/or having, or at risk of developing, a viral infection (e.g., a coronavirus infection) and/or bacterial infection can be treated with one or more inhibitors of a CRM1 polypeptide and one or more salicylates. In some cases, dogs, cats, horses, cows, pigs, sheep, mice, and rats having cancer and/or having, or at risk of developing, a viral infection (e.g., a coronavirus infection) and/or bacterial infection can be treated with one or more inhibitors of a CRM1 polypeptide and one or more salicylates as described herein.

When treating a mammal (e.g., a human) having a cancer as described herein, the cancer can be any appropriate cancer. In some cases, a cancer can include one or more solid tumors. In some cases, a cancer can be a hematologic cancer. Examples of cancers that can be treated as described herein include, without limitation, diffuse large B-cell lymphomas (DLBCLs), T-cell lymphomas (TCLs; e.g., mycosis fungoides), mantle cell lymphomas (MCLs), NHLs, plasmacytomas, Hodgkin lymphomas, MM, lymphoplasmacytic lymphomas, sarcomas (e.g., synovial sarcomas and liposarcomas), small lymphocytic lymphomas, chronic lymphocytic leukemias, acute myelogenous leukemias (AMLs), chronic myelogenous leukemias, myeloproliferative syndromes, myelodysplastic syndromes, splenic marginal zone lymphomas, MALT lymphomas, acute lymphoblastic leukemias (ALLs), chronic myelomonocytic leukemias, plasmacytic leukemias, NK cell leukemias, monoclonal gammopathies of undetermined significance, monoclonal b cell lymphocytoses, LGL leukemias, neutrophilic leukemias, myelofibrosis, polycythemia veras, myeloproliferative syndromes (e.g., essential thrombocythemias), dendritic cell cancers, histiocytic neoplasms (e.g., Langerhans cell histiocytosis), myelodysplastic syndromes (e.g., Erdheim Chester disease and Rosai-Dorfman diseas), CNS lymphomas, Bing Neel syndromes, head and neck cancers, lung cancers, breast cancers, pancreatic cancers, esophageal cancers, gastric cancers, small intestinal cancers, colon cancers, rectal cancers, anal cancers, melanomas (e.g., mucosal melanomas), skin cancers (e.g., squamous cell carcinomas and basal cell carcinomas), sarcomas, lipomas, liposarcomas, brain cancers (e.g., oligodendrogliomas, glioblastoma multiformes, and meningiomas), ovarian cancers, fallopian tube cancers, uterine cancers, cervical cancers, vaginal cancers, kidney cancers, prostate cancers, penile cancers, testicular cancers, leydig cell cancers, hepatocellular carcinomas, gallbladder cancers, biliary duct system cancers, heart cancers (e.g., angiosarcoma, and myxoma, rhabdomyosarcoma, mesothelioma, leiomyosarcoma, angiosarcoma, and angiomyolipoma), connective tissue cancers, thyroid cancers, parathyroid cancers, pituitary gland cancers, adrenal gland cancers, neuroendocrine cancers, carcinoid cancers, pheochromocytoma, insulinoma, gastrinoma, neuroblastoma, endocrine gland cancers, and exocrine gland cancers.

Any appropriate method can be used to identify a mammal (e.g., a human) having cancer. For example, imaging techniques and/or biopsy techniques can be used to identify mammals (e.g., humans) having cancer.

Once identified as having a cancer, a mammal (e.g., a human) can be administered, or instructed to self-administer, one or more inhibitors of a CRM1 polypeptide and one or more salicylates.

When treating a mammal (e.g., a human) having, or at risk of developing, a viral infection (e.g., a coronavirus infection) as described herein, the viral infection can be caused by any type of virus. In some cases, a virus whose infections can be treated as described herein can be a coronavirus (e.g., a beta-coronavirus). Examples of viruses whose infections can be treated as described herein include, without limitation, SARS-CoV, HCoV NL63, HKU1, MERS-CoV, SARS-CoV-2, influenza viruses (e.g., influenza A, influenza B, influenza C, and influenza D), respiratory syncytial viruses (RSV), human immunodeficiency viruses (HIV), and those described in Table 3-1 of “Learning from SARS: Preparing for the Next Disease Outbreak: Workshop Summary.” Institute of Medicine (US) Forum on Microbial Threats; Knobler S, Mahmoud A, Lemon S, et al., editors. Washington (DC): National Academies Press (US); 2004.

Any appropriate method can be used to identify a mammal as having, or as being at risk of developing, a viral infection (e.g., a coronavirus infection) (and/or a bacterial infection). In some cases, the presence or absence of nucleic acid from a viral genome (e.g., a coronavirus genome) in a sample obtained from a mammal can be used to identify the mammal as having, or as being at risk of developing, a viral infection. For example, the presence of nucleic acid from a viral genome in a sample obtained from a mammal can indicate that the mammal has, or is at risk of developing, a viral infection. In some cases, the presence or absence of one or more polypeptides encoded by nucleic acid in a viral genome (e.g., a coronavirus genome) in a sample obtained from a mammal can be used to identify the mammal as having, or as being at risk of developing, a viral infection. For example, the presence of one or more polypeptides encoded by nucleic acid from a viral genome in a sample obtained from a mammal can indicate that the mammal has, or is at risk of developing, a viral infection. Any appropriate sample can be used to detect the presence or absence of nucleic acid in a viral genome and/or the presence or absence of one or more polypeptides encoded by nucleic acid in a viral genome. In some cases, a sample can be a biological sample. In some cases, a sample can contain one or more cells. In some cases, a sample can contain one or more biological molecules (e.g., nucleic acids such as DNA and RNA, polypeptides, carbohydrates, lipids, hormones, and/or metabolites). Examples of samples that can be obtained from a mammal and can be used to detect the presence or absence of nucleic acid in a coronavirus genome and/or the presence or absence of one or more polypeptides encoded by nucleic acid in a coronavirus genome as described herein include, without limitation, tissue samples (e.g., lung tissues such as those obtained by biopsy), fluid samples (e.g., whole blood, serum, plasma, urine, and saliva), and cellular samples (e.g., nasopharyngeal samples, and buccal samples). A sample can be a fresh sample or a fixed sample (e.g., a formaldehyde-fixed sample or a formalin-fixed sample). In some cases, a sample can be a processed sample (e.g., an embedded sample such as a paraffin or OCT embedded sample). In some cases, one or more biological molecules can be isolated from a sample. For example, nucleic acid (e.g., DNA and RNA such as messenger RNA (mRNA)) can be isolated from a sample and can be used to detect the presence or absence of nucleic acid in a coronavirus genome and/or the presence or absence of one or more polypeptides encoded by nucleic acid in a coronavirus genome. For example, one or more polypeptides can be isolated from a sample and can be used to detect the presence or absence of nucleic acid in a coronavirus genome and/or the presence or absence of one or more polypeptides encoded by nucleic acid in a coronavirus genome. Any appropriate method can be used to detect the presence or absence of nucleic acid in a coronavirus genome and/or the presence or absence of one or more polypeptides encoded by nucleic acid in a coronavirus genome. In some cases, polymerase chain reaction (PCR)-based techniques such as quantitative reverse transcription (RT)-PCR (qPCR) techniques, RNA in situ hybridization (ISH), and/or RNA sequencing can be used to detect the presence or absence of nucleic acid in a coronavirus genome. In some cases, immunoassays (e.g., immunohistochemistry (IHC) techniques, and western blotting techniques), mass spectrometry techniques (e.g., proteomics-based mass spectrometry assays or targeted quantification-based mass spectrometry assays), and/or enzyme-linked immunosorbent assays (ELISAs) can be used to detect the presence or absence of one or more polypeptides encoded by nucleic acid in a coronavirus genome.

Once (a) identified as having, or as being at risk of developing, a viral infection (e.g., a coronavirus infection) (and/or a bacterial infection), a mammal (e.g., a human) can be administered, or instructed to self-administer, one or more inhibitors of a CRM1 polypeptide and one or more salicylates.

In some cases, administering one or more inhibitors of a CRM1 polypeptide in combination with one or more salicylates can be effective to arrest the cell cycle of a cell (e.g., a cell in a mammal such as a human). For example, administering one or more inhibitors of a CRM1 polypeptide in combination with one or more salicylates can be effective to arrest the cell cycle of a cell at the G0-G1 phase. For example, administering one or more inhibitors of a CRM1 polypeptide in combination with one or more salicylates can be effective to arrest the cell cycle of a cell at the S phase. For example, one or more inhibitors of a CRM1 polypeptide in combination with one or more salicylates can be used to reduce proliferation of a cell (e.g., a cell in a mammal such as a human) by, for example, 10, 20, 30, 40, 50, 60, 70, 80, 90, 95, or more percent.

In some cases, administering one or more inhibitors of a CRM1 polypeptide in combination with one or more salicylates can be effective to reduce a level of one or more polypeptides associated with a DNA damage repair pathway within a cell (e.g., a cell in a mammal such as a human). For example, one or more inhibitors of a CRM1 polypeptide in combination with one or more salicylates can be administered to a mammal in need thereof (e.g., a mammal having cancer) to reduce a level of one or more polypeptides associated with a DNA damage repair pathway within a cell (e.g., a cell in a mammal such as a human) by, for example, 10, 20, 30, 40, 50, 60, 70, 80, 90, 95, or more percent. A DNA damage repair pathway can be any appropriate DNA damage repair pathway (e.g., DNA homologous recombination (HR)). Examples of polypeptides associated with a DNA damage repair pathway whose level can be reduced by administering one or more inhibitors of a CRM1 polypeptide in combination with one or more salicylates include, without limitation, Rad51 polypeptides, BRCA1 polypeptides, BRCA2 polypeptides, CDK12 polypeptides, CHEK1 polypeptides, PALB2 polypeptides, TP53 polypeptides, ATM polypeptides, ATR polypeptides, RAD51AP1, BLM, and RAD9A.

In some cases, administering one or more inhibitors of a CRM1 polypeptide in combination with one or more salicylates can be effective to reduce a level of one or more cytokines within a cell (e.g., a cell in a mammal such as a human). For example, one or more inhibitors of a CRM1 polypeptide in combination with one or more salicylates can be administered to a mammal in need thereof (e.g., a mammal having cancer) to reduce a level of one or more cytokines within a cell (e.g., a cell in a mammal such as a human) by, for example, 10, 20, 30, 40, 50, 60, 70, 80, 90, 95, or more percent. A cytokine can be any appropriate cytokine (e.g., chemokine, interferon, interleukin, lymphokine, or tumour necrosis factor). In some cases, a cytokine can be an inflammatory cytokine. Examples of cytokines whose level can be reduced by administering one or more inhibitors of a CRM1 polypeptide in combination with one or more salicylates include, without limitation, interleukin 1 beta (IL-1beta), interleukin 10 (IL-10), granulocyte macrophage colony-stimulating factor (GM-CSF), interleukin 8 (IL-8), interleukin 5 (IL-5), interferon gamma (IFN-gamma), tumor necrosis factor alpha (TNF-alpha), interleukin 2 (IL-2), interleukin 4 (IL-4), and interleukin 6 (IL-6).

An inhibitor of a CRM1 polypeptide can be any appropriate inhibitor of a CRM1 polypeptide. An inhibitor of a CRM1 polypeptide can be an inhibitor of CRM1 polypeptide expression or an inhibitor of CRM1 polypeptide activity. Examples of compounds that can reduce polypeptide activity include, without limitation, antibodies (e.g., neutralizing antibodies) and small molecules. Examples of compounds that can reduce polypeptide expression include, without limitation, nucleic acid molecules designed to induce RNA interference (e.g., a siRNA molecule or a shRNA molecule), antisense molecules, and miRNAs. In some cases, an inhibitor of a CRM1 polypeptide can inhibit nuclear export (e.g., leucine-rich nuclear export signal (NES)-dependent nuclear export) of one or more biological molecules (e.g., proteins, rRNAs, snRNAs, and mRNAs) from a cell. In some cases, an inhibitor of a CRM1 polypeptide can be a selective inhibitor of nuclear exports (SINE). In some cases, an inhibitor of a CRM1 polypeptide can bind (e.g., covalently bind) to a CRM1 polypeptide. Examples of inhibitors of a CRM1 polypeptide that can be used in combination with one or more salicylates as described herein include, without limitation, selinexor (KPT-330), leptomycin B, KPT-185, KPT-276, eltanexor (KPT-8602), piperlongumine, verdinexor (KPT-335), valtrate, and ratjadone C. In some cases, an inhibitor of a CRM1 polypeptide can be as described in Table 1.

TABLE 1
Inhibitors of a CRM1 polypeptide.

| Inhibitor | Chemical Structure | Chemical Name | Source |
|---|---|---|---|
| selinexor (KPT-330) | 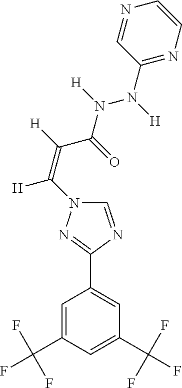 [SMILES: O=C(/C=C\n1cnc(-c2cc(C(F)(F)F)cc(C(F)(F)F)c2)n1)NNc1cnccn1] | (Z)-3-[3-[3,5-bis (trifluoromethyl)phenyl]- 1,2,4-triazol-1-yl]-N′- pyrazin-2-ylprop-2- enehydrazide | National Center for Biotechnology Information (NCBI) PubChem Database. Selinexor, CID = 71481097, pubchem.ncbi.nlm.nih.gov/ compound/71481097 |
| leptomycin B | 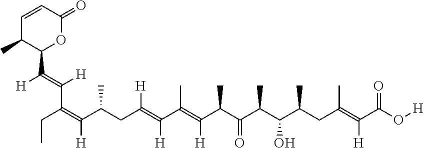 [SMILES: CCC(=C/[C@H](C)C/C=C/C(C)=C/[C@@H](C)C(=O)[C@@H](C)[C@@H](O)[C@@H](C)C/C(C)=C/C(=O)O)/C=C/[C@@H]1OC(=O)C=C[C@@H]1C] | (2E,5S,6R,7S,9R,10E, 12E,15R,16Z,18E)-17- ethyl-6-hydroxy-3,5,7,9, 11,15-hexamethyl-19- [(2S,3S)-3-methyl-6-oxo- 2,3-dihydropyran-2-yl]-8- oxononadeca-2,10,12,16, 18-pentaenoic acid | NCBI PubChem Database. Leptomycin B, CID = 6917907, pubchem.ncbi.nlm.nih.gov/ compound/6917907 |
| KPT-185 | 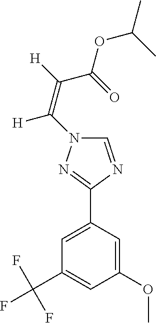 [SMILES: COc1cc(-c2ncn(/C=C\C(=O)OC(C)C)n2)cc(C(F)(F)F)c1] | propan-2-yl (Z)-3-[3-[3- methoxy-5- (trifluoromethyl)phenyl]- 1,2,4-triazol-1-yl]prop-2- enoate | NCBI PubChem Database. NLNGWFLRRRYNIL- PLNGDYQASA-N, CID = 53495165, pubchem.ncbi.nlm.nih.gov/ compound/53495165 |
| KPT-276 | 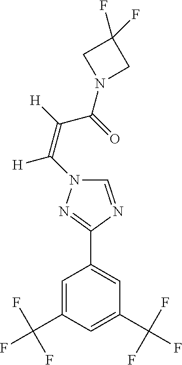 [SMILES: O=C(/C=C\n1cnc(-c2cc(C(F)(F)F)cc(C(F)(F)F)c2)n1)N1CC(F)(F)C1] | (Z)-3-[3-[3,5-bis (trifluoromethyl)phenyl]- 1,2,4-triazol-1-yl]-1-(3,3- difluoroazetidin-1-yl)prop- 2-en-1-one | NCBI PubChem Database. JCHAWRDHMUSLMM- UPHRSURJSA-N, CID = 71496742, pubchem.ncbi.nlm.nih.gov/ compound/71496742 |
| KPT-335 | 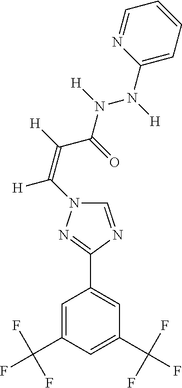 [SMILES: O=C(/C=C\n1cnc(-c2cc(C(F)(F)F)cc(C(F)(F)F)c2)n1)NNc1ccccn1] | (Z)-3-[3-[3,5-bis (trifluoromethyl)phenyl]- 1,2,4-triazol-1-yl]-N′- pyridin-2-ylprop-2- enehydrazide | NCBI PubChem Database. Verdinexor, CID = 71492799, pubchem.ncbi.nlm.nih.gov/ compound/71492799 |
| KPT-8602 | 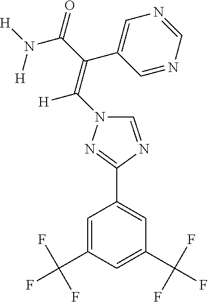 [SMILES: NC(=O)/C(=C/n1cnc(-c2cc(C(F)(F)F)cc(C(F)(F)F)c2)n1)c1cncnc1] | (E)-3-[3-[3,5-bis (trifluoromethyl)phenyl]- 1,2,4-triazol-1-yl]-2- pyrimidin-5-ylprop-2- enamide | NCBI PubChem Database. Eltanexor, CID = 86345880, pubchem.ncbi.nlm.nih.gov/ compound/86345880 |
| Valtrate | 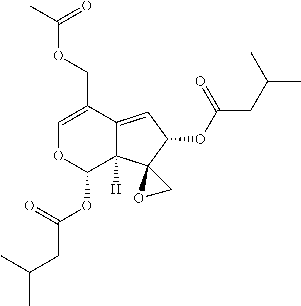 [SMILES: CC(=O)OCC1=CO[C@@H](OC(=O)CC(C)C)[C@H]2C1=C[C@H](OC(=O)CC(C)C)[C@]21CO1] | [(1S,6S,7R,7aS)-4- (acetyloxymethyl)-1- (3-methylbutanoyloxy) spiro[6,7a-dihydro-1H- cyclopenta[c]pyran-7,2′- oxirane]-6-yl] 3- methylbutanoate | NCBI PubChem Database. Valtrate, CID = 442436, pubchem.ncbi.nlm.nih.gov/ compound/442436 |
| Ratjadone C | 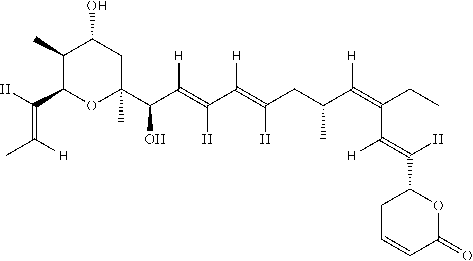 [SMILES: C/C=C/[C@@H]1O[C@](C)([C@H](O)/C=C/C=C/C[C@@H](C)/C=C(\C=C\[C@H]2CC=CC(=O)O2)CC)C[C@@H](O)[C@@H]1C] | (2R)-2- [(1E,3Z,5R,7E,9E,11R)- 3-ethyl-11-hydroxy-11- [(2S,4R,5S,6S)-4- hydroxy-5-methyl-6- [(E)-prop-1-enyl]oxan- 2-yl]-5-methylundeca- 1,3,7,9-tetraenyl]-2,3- dihydropyran-6-one | NCBI PubChem Database. QHBAIVURIOKLSG- RVNOVNSXSA-N, CID = 97947825, pubchem.ncbi.nlm.nih.gov/ compound/97947825 |

In cases where an inhibitor of a CRM1 polypeptide is an inhibitor of CRM1 polypeptide activity, administering one or more inhibitors of a CRM1 polypeptide in combination with one or more salicylates can be effective to reduce or eliminate the level of CRM1 polypeptides in a cell (e.g., a cell in a mammal such as a human). A reduced level of CRM1 polypeptides refers to any level of CRM1 polypeptides that is lower than the median level of CRM1 polypeptides typically observed in a cell (e.g., a control cell) from one or more healthy mammals (e.g., healthy humans). Control cells can include, without limitation, cells from mammals that do not have cancer, cell lines originating from mammals that do not have cancer, and non-tumorigenic cell lines. An eliminated level of CRM1 polypeptides refers to any non-detectable level of CRM1 polypeptides. Any appropriate method can be used to determine whether or not a cell has a reduced or eliminated level of CRM1 polypeptides. For example, western blotting, reverse-transcription polymerase chain reaction (RT-PCR), spectrometry methods (e.g., high-performance liquid chromatography (IPLC) and liquid chromatography-mass spectrometry (LC/MS)), enzyme-linked immunosorbent assay (ELISA), immunohistochemistry, immunofluorescence microscopy, CO-Detection by indEXing (CODEX) imaging, and/or mass cytometry (CyTOF) can be used to determine whether or not a cell contains a reduced or eliminated levels of CRM1 polypeptides. For example, administering one or more inhibitors of a CRM1 polypeptide in combination with one or more salicylates can be effective to reduce the expression level of CRM1 polypeptides in a cell (e.g., a cell in a mammal such as a human) by, for example, 10, 20, 30, 40, 50, 60, 70, 80, 90, 95, or more percent.

A salicylate can be any appropriate salicylate. A salicylate (e.g., a salt or an ester of a salicylic acid) can include any appropriate type of salicylic acid (e.g., acetylsalicylic acid). In some cases, a salicylate can be a compound that includes compound which has a salicylate moiety. In some cases, a salicylate can have nonsteroidal anti-inflammatory drug (NSAID) activity. Examples of salicylates that can be used in combination with one or more inhibitors of a CRM1 polypeptide as described herein include, without limitation, aspirin, choline salicylate, sodium salicylate, acetyl salicylate, choline magnesium salicylate (e.g., choline magnesium trisalicylate), and salts thereof.

One or more inhibitors of a CRM1 polypeptide and one or more salicylates can be administered to a mammal having a cancer at the same time or independently. When one or more inhibitors of a CRM1 polypeptide and one or more salicylates are administered at the same time, one or more inhibitors of a CRM1 polypeptide and one or more salicylates can be administered as separate compositions administered at the same time or can be present in a single composition.

In some cases, one or more inhibitors of a CRM1 polypeptide and one or more salicylates can be administered to a mammal having a cancer as the sole active ingredients used to treat a cancer.

In some cases, one or more inhibitors of a CRM1 polypeptide and one or more salicylates can be administered to a mammal having a cancer as a combination therapy with one or more additional cancer treatments used to treat a cancer. For example, a combination therapy used to treat a cancer can include administering to the mammal (e.g., a human) one or more inhibitors of a CRM1 polypeptide and one or more salicylates described herein and one or more cancer treatments such as surgery, chemotherapy, radiation, targeted therapy (e.g., PARP inhibitors such as olaparib, rucaparib, niraparib, talazoparib, veliparib, pamiparib, and iniparib), and/or immunotherapy. In cases where one or more inhibitors of a CRM1 polypeptide and one or more salicylates described herein are used in combination with one or more additional cancer treatments, the one or more additional cancer treatments can be administered at the same time or independently. For example, one or more inhibitors of a CRM1 polypeptide and one or more salicylates described herein can be administered first, and the one or more additional cancer treatments can be administered second, or vice versa.

In some cases, one or more inhibitors of a CRM1 polypeptide and/or one or more salicylates can be formulated into a composition (e.g., pharmaceutically acceptable composition) for administration to a mammal having cancer. For example, a therapeutically effective amount of one or more inhibitors of a CRM1 polypeptide and/or of one or more salicylates can be formulated together with one or more pharmaceutically acceptable carriers (additives) and/or diluents. A pharmaceutical composition can be formulated for administration in any appropriate dosage form. Examples of dosage forms include solid or liquid forms including, without limitation, gums, capsules, tablets (e.g., chewable tablets, and enteric coated tablets), suppository, liquid, enemas, suspensions, solutions (e.g., sterile solutions), sustained-release formulations, delayed-release formulations, pills, powders, gels, creams, ointments, and granules. Pharmaceutically acceptable carriers, fillers, and vehicles that may be used in a pharmaceutical composition described herein include, without limitation, ion exchangers, alumina, aluminum stearate, lecithin, serum proteins such as human serum albumin, buffer substances such as phosphates, glycine, sorbic acid, potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol such as Vitamin E TPGS, sodium carboxymethylcellulose, polyacrylates, waxes, polyethylene-polyoxypropylene-block polymers, and wool fat.

A composition (e.g., a pharmaceutical composition) containing one or more inhibitors of a CRM1 polypeptide and/or one or more salicylates can be designed for oral or parenteral (including subcutaneous, intratumoral, intramuscular, intravenous, topical, and intradermal) administration. When being administered orally, a pharmaceutical composition containing one or more inhibitors of a CRM1 polypeptide and/or one or more salicylates can be in the form of a pill, syrup, gel, liquid, flavored drink, tablet, or capsule. Compositions suitable for parenteral administration include aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

A composition (e.g., a pharmaceutical composition) containing one or more inhibitors of a CRM1 polypeptide and/or one or more salicylates can be administered locally or systemically. For example, a composition containing one or more inhibitors of a CRM1 polypeptide and/or one or more salicylates can be administered systemically by an oral administration or by injection to a mammal (e.g., a human).

Effective doses of one or more inhibitors of a CRM1 polypeptide and/or one or more salicylates can vary depending on the severity of the cancer, the route of administration, the age and general health condition of the subject, excipient usage, the possibility of co-usage with other therapeutic treatments such as use of other agents, the specific CRM1 inhibitor being used, and/or the judgment of the treating physician.

An effective amount of a composition containing one or more inhibitors of a CRM1 polypeptide and/or one or more salicylates can be any amount that can treat the cancer without producing significant toxicity to the mammal. An effective amount of an inhibitor of a CRM1 polypeptide can be any appropriate amount. In some cases, an effective amount of an inhibitor of a CRM1 polypeptide such as selinexor can be from about 35 mg/kg body weight of a mammal to about 45 mg/kg body weight of a mammal. In some cases, an effective amount of an inhibitor of a CRM1 polypeptide such as selinexor can from about 0.01 nM to about 2.5 μM plasma concentration. For example, an effective amount of selinexor can be from about 0.25 μM to about 1.24 μM. An effective amount of a salicylate can be any appropriate amount. In some cases, an effective amount of a salicylate can be from about 0.0001 mM to about 10 mM plasma concentration. For example, an effective amount of a salicylate can be from about 0.1 μM to about 10 mM (e.g., from about 1 μM to about 3 mM). The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition (e.g., a cancer) may require an increase or decrease in the actual effective amount administered.

The frequency of administration of a composition containing one or more inhibitors of a CRM1 polypeptide and/or one or more salicylates can be any frequency that can treat the cancer without producing significant toxicity to the mammal. For example, the frequency of administration can be from about three times a day to about once a week, from about twice a day to about twice a week, or from about once a day to about twice a week. The frequency of administration can remain constant or can be variable during the duration of treatment. A course of treatment with a composition containing one or more inhibitors of a CRM1 polypeptide and/or one or more salicylates can include rest periods. For example, a composition containing one or more inhibitors of a CRM1 polypeptide and/or one or more salicylates can be administered daily over a two-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., a cancer) may require an increase or decrease in administration frequency.

An effective duration for administering a composition containing one or more inhibitors of a CRM1 polypeptide and/or one or more salicylates can be any duration that treat the cancer without producing significant toxicity to the mammal. For example, the effective duration can vary from several days to several weeks, months, or years. In some cases, the effective duration for the treatment of a cancer can range in duration from about one month to about 10 years. Multiple factors can influence the actual effective duration used for a particular treatment. For example, an effective duration can vary with the frequency of administration, effective amount, use of multiple treatment agents, route of administration, and severity of the condition being treated.

In some cases, the number of cancer cells present within a mammal, and/or the severity of one or more symptoms of the cancer being treated can be monitored. Any appropriate method can be used to determine whether or not the number of cancer cells present within a mammal is reduced. For example, imaging techniques can be used to assess the number of cancer cells present within a mammal.

In some cases, the materials and methods described herein also can be used to treat other diseases or disorders that are characterized by increased expression of CRM1 polypeptides and/or increased activity of CRM1 polypeptides.

The invention will be further described in the following examples, which do not limit the scope of the invention described in the claims.

### EXAMPLES

### Example 1: Salicylates Enhance Antitumor Activity of CRM1 Inhibitors In Vitro

Aspirin and choline salicylate are nonsteroidal anti-inflammatory drugs (NSAID) under the salicylate family and were used to assess the synergy between salicylates and selinexor.

### Methods

### Cell Lines

All cell lines were obtained from commercial vendors (ATCC or DSMZ), which includes mantle cell lymphoma (MCL) cell lines: Jeko-1, Mino, JVM-2; T-cell lymphoma (TCL) cell lines: SU-DHL-1, Karpas-299, SR-786 and HUT-78; and diffuse large B-cell lymphoma cell lines: OCI-Ly1, SU-DHL-2, OCI-Ly3, SU-DHL-6; Waldenstrom macroglobulinemia (WM) cell lines: BCWM, MWCL, RPCI; sarcoma cell lines: FUJI, SW872, and pancreatic cancer cell lines: PANC-1.

### Cell Culture

WM, TCL, and MCL cell lines were cultured in RPMI media supplemented with 10% fetal bovine serum while DLBCL cell lines were cultured in IMDM media with 10% human serum (Sigma). Pancreatic cancer and sarcoma cell lines were cultured in DMEM with 10% fetal bovine serum. All cell lines were assessed for their viability before use in experiments. Only cells with the viability of 85% or greater were used.

### Western Blot

After treating with salicylates (concentrations ranging from 1-5 mM) and CRM 1 inhibitors (concentrations ranging from 0.1-2 μM) as monotherapy and in combination for 24 hours, cells were extracted in lysis buffer containing protease inhibitor, PMSF, and HALT phosphatase inhibitor for total cellular proteins. The cell lysates were diluted in Laemmli sample buffer supplemented with beta-mercaptoethanol and the proteins were resolved in a precast 4-15% gradient Mini SDS gels (Bio-Rad) by electrophoresis, and then transferred to PVDF membranes. The membranes were blocked with LI-COR ODB/PBS buffer and probed with primary antibodies to CRM1 and ikB (rabbit antibody from Cell Signaling; catalong number 46249) or mouse antibody for Actin (from santa cruz biotechnology), followed with fluorescent secondary antibodies anti-rabbit IRDye 800CW or anti-mouse IRDye 700CW (LI-COR) for one hour. The membranes were imaged on a LI-Cor Odyssey CLX imager.

### Treatment for Apoptosis and Proliferation Assay

To assess apoptosis and cellular proliferation, all cell lines were treated with CRM 1 inhibitors (concentration ranging from 0.05-2 μM) and salicylates at concentrations ranging from (0.001-10 mM) for 48 hours before analysis was performed. DMSO was used to substitute the drug in controls.

### Apoptosis Assay and Cell Cycle Analysis

After 48 hours incubation, cells were washed with Annexin buffer solution and stained with both propidium iodide (PI) and FITC-Annexin V (Life Technologies), and assayed on a BD Accuri flow cytometer (BD Biosciences). Apoptosis results were analyzed with BD CellQuest software. For cell cycle analysis, Jeko-1 cell line was intubated with CRM 1 inhibitors (concentrations ranging from 0.1-0.5p M) and salicylates (concentration at 3 mM) and Ly-1 cell line was intubated with CRM 1 inhibitors (concentrations ranging from 0.1-2.0 μM) and salicylates (concentration at 0.5-5 mM) for 24 and 48 hours and then fixed with 95% cold ethanol and kept at 4 C° for 24 h and subsequently underwent cell cycle analysis after PI staining and analysis was performed using BD FACS caliber flow cytometer (BD Biosciences) and FlowJo software (Tree Star).

### Proliferation Assay

According to a standard proliferation procedure, cells were seeded in a 96 well plate with respective concentrations of CRM 1 inhibitors and salicylates as mentioned above. After 48 h of incubation, ³[H] labeled thymidine (CMP) was added. The plate was harvested after another 18 hr of incubation. ³[H]-thymidine uptake was measured on a MicroBeta workstation (Perkin Elmer).

In some cases, the methods can be performed as described elsewhere (see, e.g., Abeykoon et al., Blood Cancer J., 9:24 (2019)).

### Results

Antitumor Effect of Selinexor in Combination with Salicylates

Significant enhancement of the antitumor effect in DLBCL cells, MCL cells, and TCL cells were observed at very low concentrations of selinexor; 0.25-0.5 μM (10-20% of the concentration being used in humans in clinical trial setting), when selinexor is combined with salicylates (in human tolerated concentrations ranging from 0.5 mM to 3 mM).

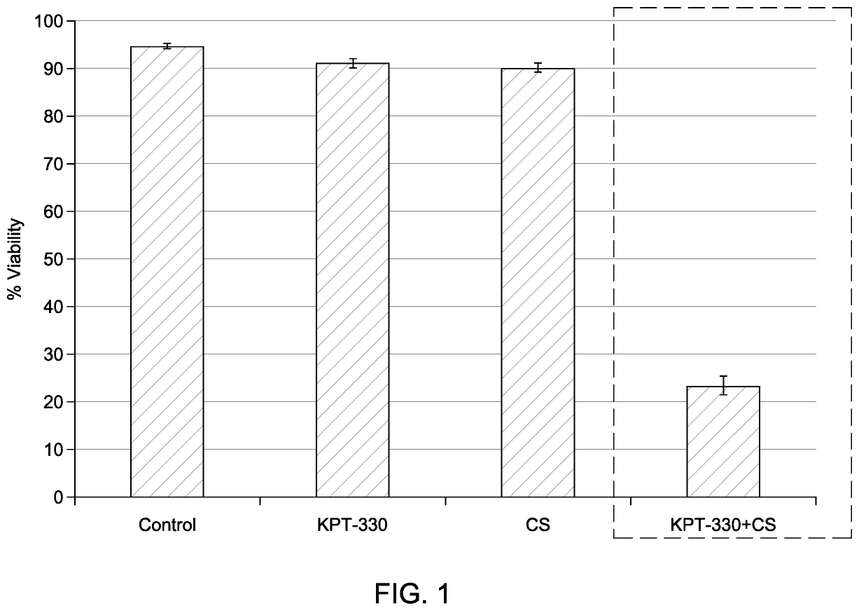

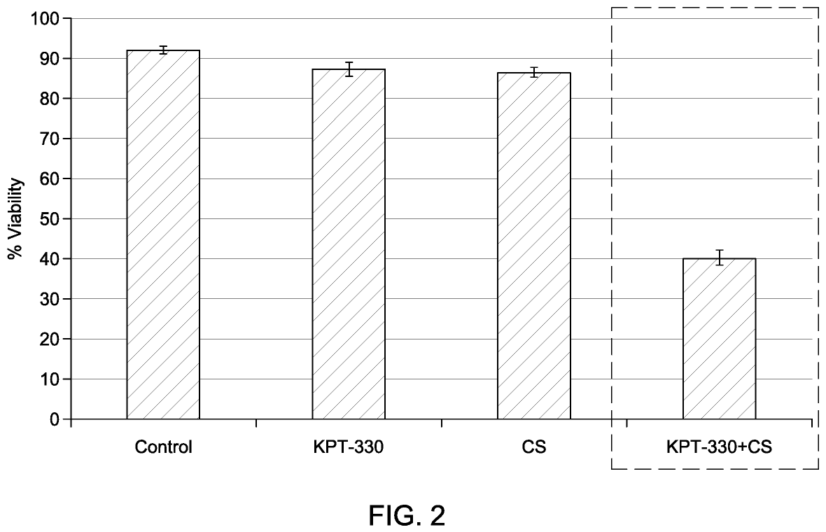

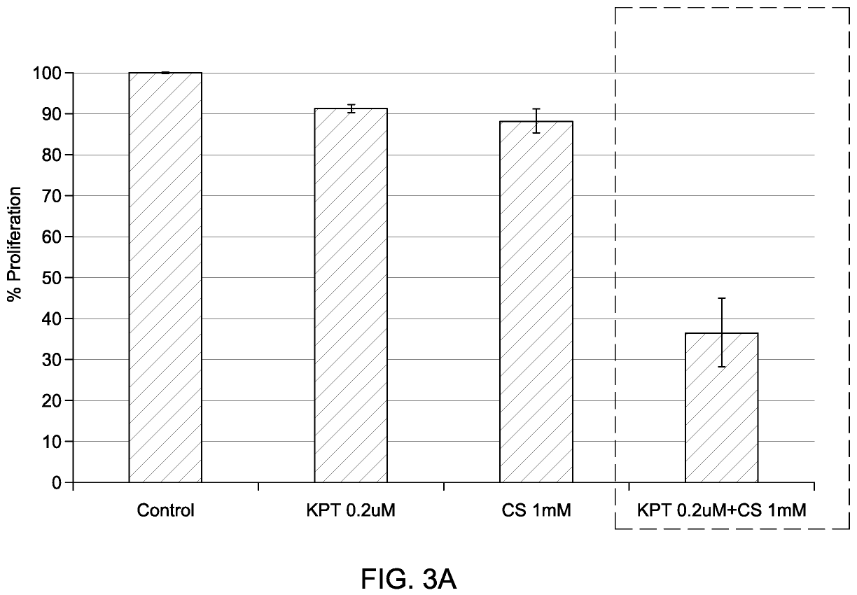

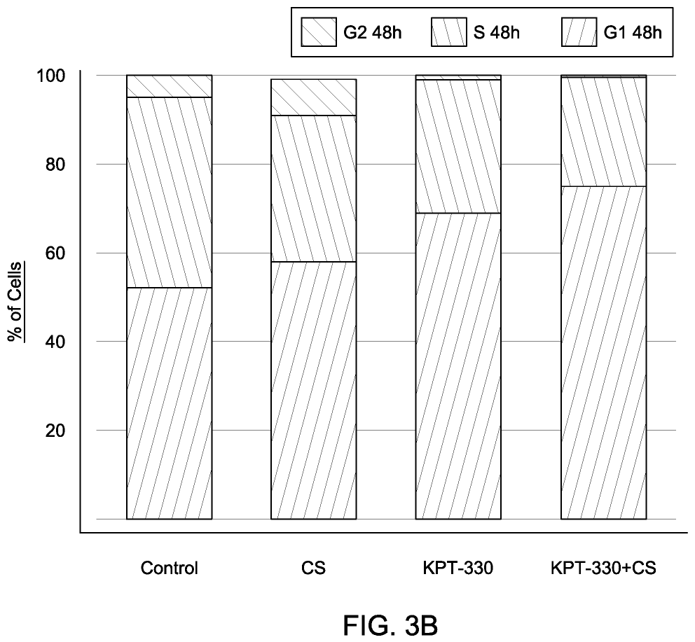

Antitumor effects evaluated included cell viability, cell proliferation, and cell cycle progression. DLBCL cells (Ly-1 cell line) treated for 72 hours with a combination drug treatment including selinexor at 0.01-2.0 μM and CS at 0.1-5 mM showed decreased viability (FIG. 1). MCL cells (Jeko-1 cell line) treated for 48 hours with a combination drug treatment including selinexor at 0.5 μM and CS at 3 mM also showed decreased viability (FIG. 2). MCL cells (Jeko-1 cell line) treated for 48 hours with a combination drug treatment including selinexor at 0.5 μM and CS at 3 mM also showed decreased cell proliferation (FIG. 3A); and also showed an increase in the number of cells with arrested cell cycle progression (FIG. 3B). The arrested in the cell-cycle in FIG. 3B is reflected by the hindered proliferation in FIG. 3A. Moreover, enhanced cell cycle arrest at G0-G1 phase was observed when selinexor was combined with salicylates (both with Aspirin or choline salicylate) compared to selinexor alone. Cell cycle was not affected by salicylates alone.

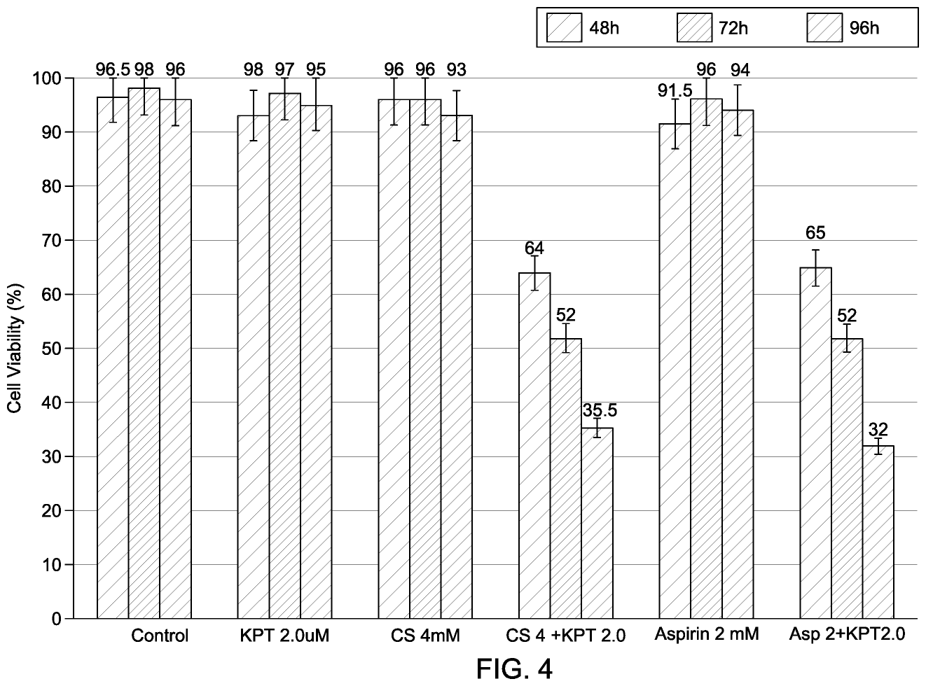

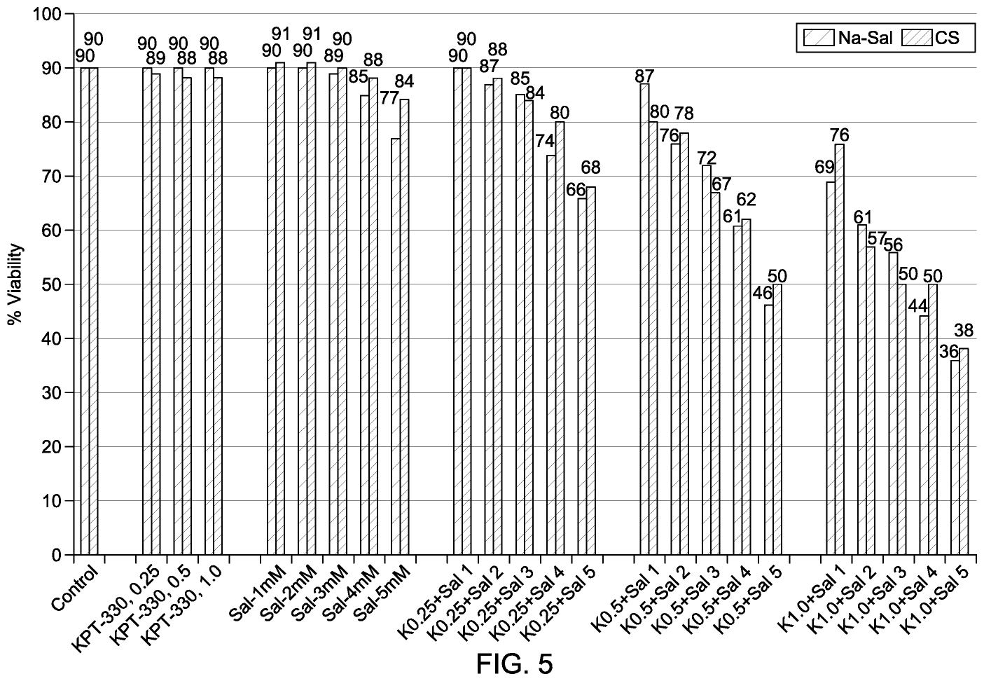

A variety of salicylates were also evaluated. Cell viability of DLBCL cells (Ly-1 cell line) treated with selinexor in combination with CS, aspirin, or sodium salicylate was examined. DLBCL cells (Ly-1 cell line) treated for 48, 72, or 96 hours with a combination drug treatment including selinexor at 2.0 μM and CS at 4 mM or aspirin at 2 mM showed decreased viability (FIG. 4). FIG. 4 includes data from representative example of the synergistic antitumor effect when selinexor is combined with CS or aspirin. Similar synergy is seen when selinexor is combined with aspirin in MCL cells and other DLBCL cell lines. DLBCL cells (Ly-1 cell line) treated for 48 hours with a combination drug treatment including selinexor at 0.25 μM, 0.5 μM, or 1.0 μM and CS at 1 mM, 2 mM, 3 mM, 4 mM, or 5 mM or sodium salicylate at 1 mM, 2 mM, 3 mM, 4 mM, or 5 mM showed decreased viability (FIG. 5). Similar augmentation of antitumor activity was observed when tumor cells were treated in combination with leptomycin B (a pure CRM1 inhibitor) or KPT-185 (an older generation of a CRM1 inhibitor) and salicylates suggesting that the mechanism underling synergy is specific for CRM1 inhibition.

Potency of Selinexor in Combination with Salicylates

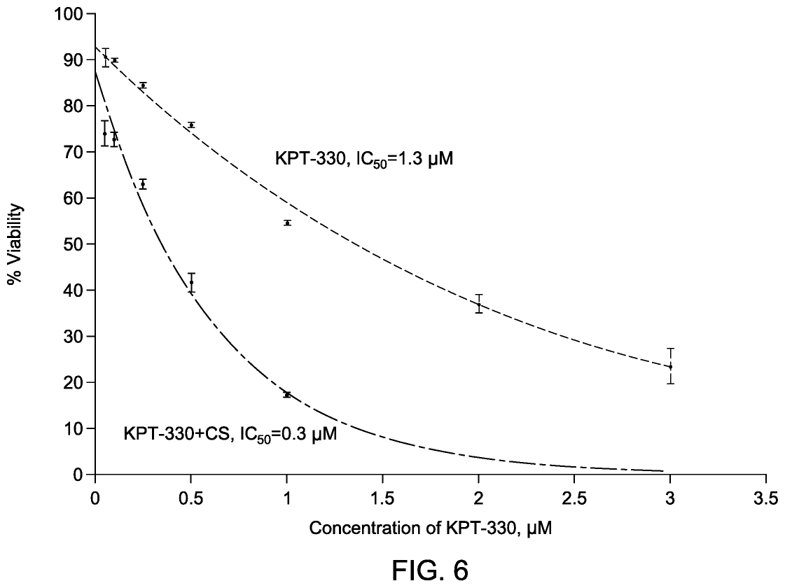

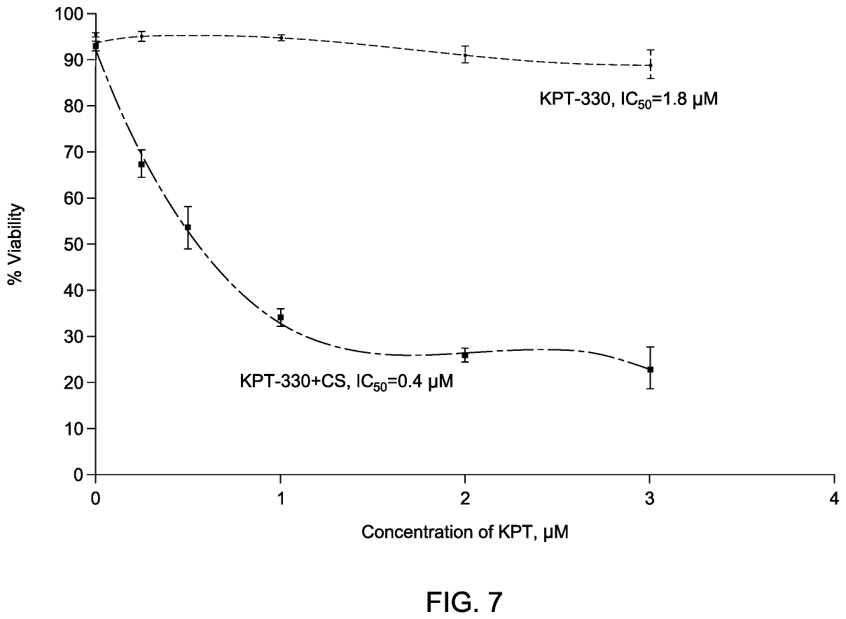

The ability of selinexor used in combination with a salicylate to decrease the IC₅₀ was evaluated using different concentrations of selinexor and using 3 mM of CS. MCL cells treated for 72 hours with selinexor alone had an IC₅₀ of 1.3 μM, while a combination drug treatment including selinexor and CS had an IC₅₀ of 0.3 μM (FIG. 6). DLBCL cells treated for 72 hours with selinexor alone had an IC₅₀ of 1.8 μM, while a combination drug treatment including selinexor and CS had an IC₅₀ of 0.4 μM (FIG. 7).

### CRM1 Protein Expression

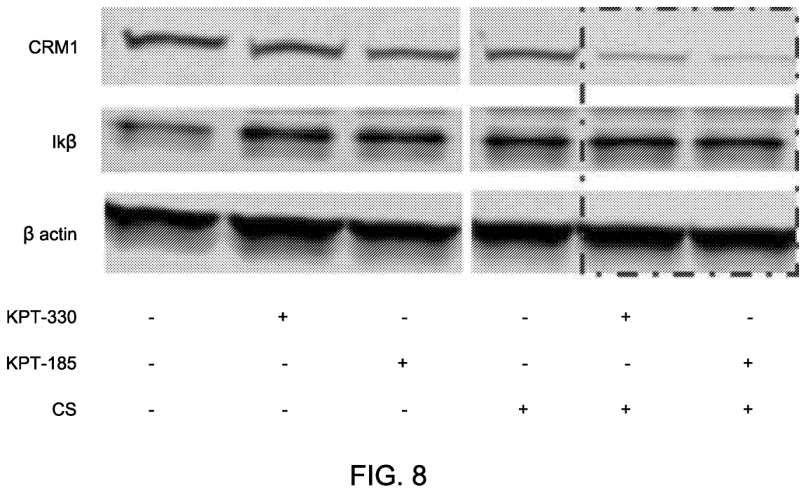

CRM1 polypeptide expression levels were evaluated in cells treated with selinexor or KPT-185 in combination with CS. The drug combination selectively decreased the expression of CRM1 polypeptides and no such effect was seen with respect to the expression of IkB (FIG. 8).

Further investigations also suggested that when selinexor is combined with salicylates, CRM1 protein expression decreased but this decrease expression was not evident with either drug alone.

Selinexor in Combination with Other Non-Salicylate NSAIDS

The specificity of the synergy between selinexor and NSAIDs was evaluated by treating cells with selinexor alone or with selinexor in combination with non-salicylate NSAIDs ketorolac and ibuprofen.

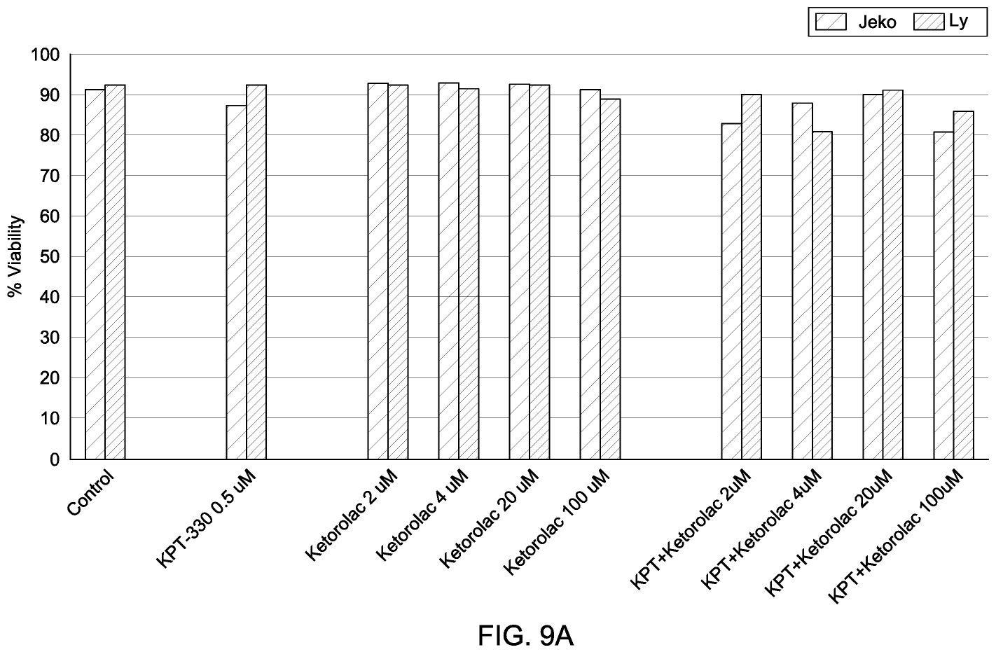

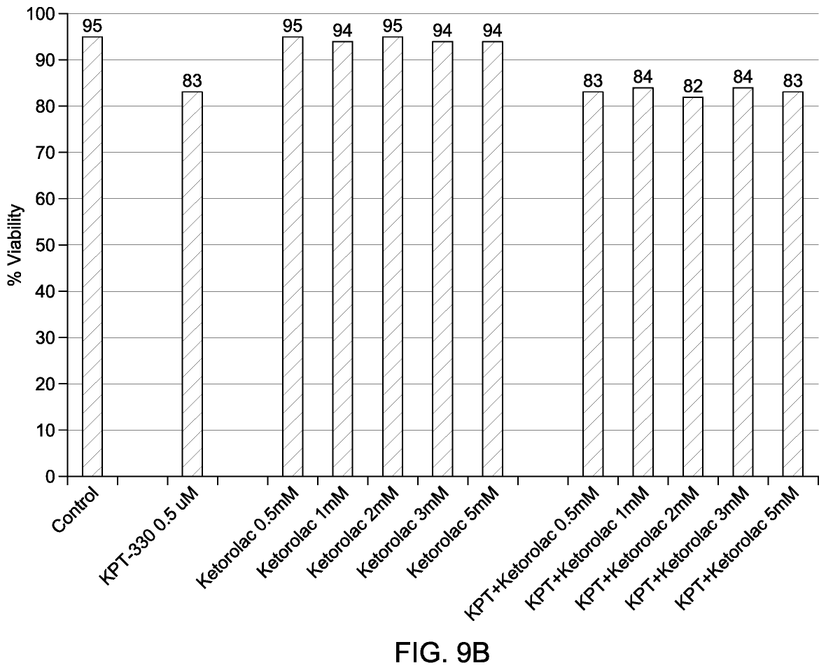

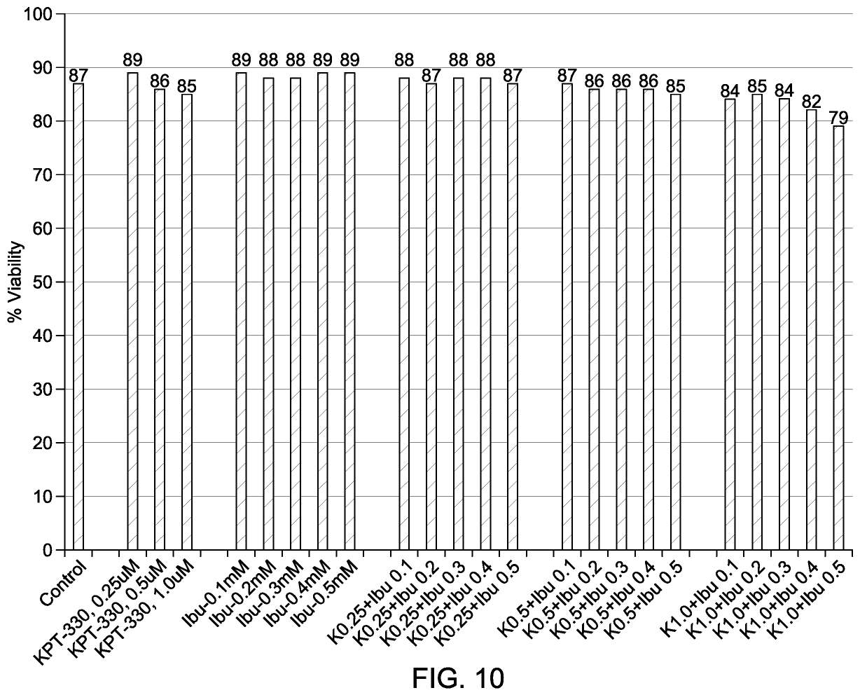

Cell viability of cancer cells treated with selinexor in combination with non-salicylate NSAIDs was examined. MCL cells or DLBCL cells treated with a combination drug treatment including selinexor at 0.5 μM and ketorolac at 2 μM, 4 μM, 20 μM, or 100 μM showed no change in cell viability (FIG. 9A). MCL cells treated with a combination drug treatment including selinexor at 0.5 μM and high concentrations of ketorolac at 0.5 mM, 1 mM, 2 mM, 3 mM, or 5 mM showed no change in cell viability (FIG. 9B). DLBCL cells treated for 72 hours with a combination drug treatment including selinexor at 0.25 μM, 0.5 μM, or 1.0 μM and ibuprofen at 1 mM, 2 mM, 3 mM, 4 mM, or 5 mM showed no change in cell viability (FIG. 10).

The effect was similar when selinexor was combined both with aspirin or choline salicylate independently at human tolerated concentrations. No synergistic antitumor activity was seen when selinexor or leptomycin B was combined with non-salicylate NSAIDs. These results suggest the synergy is not NSAID drug class specific but it is salicylate specific. Further, salicylates or non-salicylate NSAIDs alone did not impose any antitumor effect in cancer cells.

Selinexor in Combination with Chemotherapeutics

Synergy between selinexor and salicylates was further evaluated by further treating cells with chemotherapeutics.

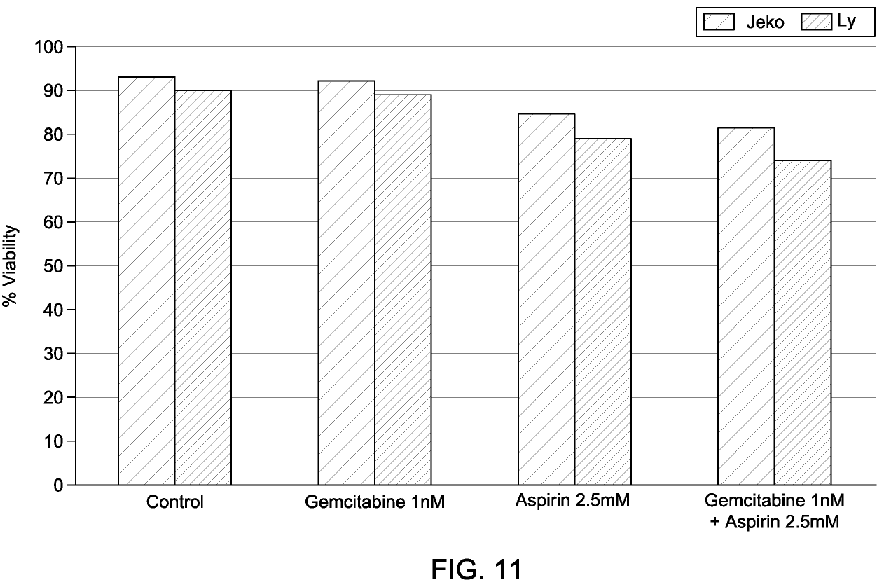

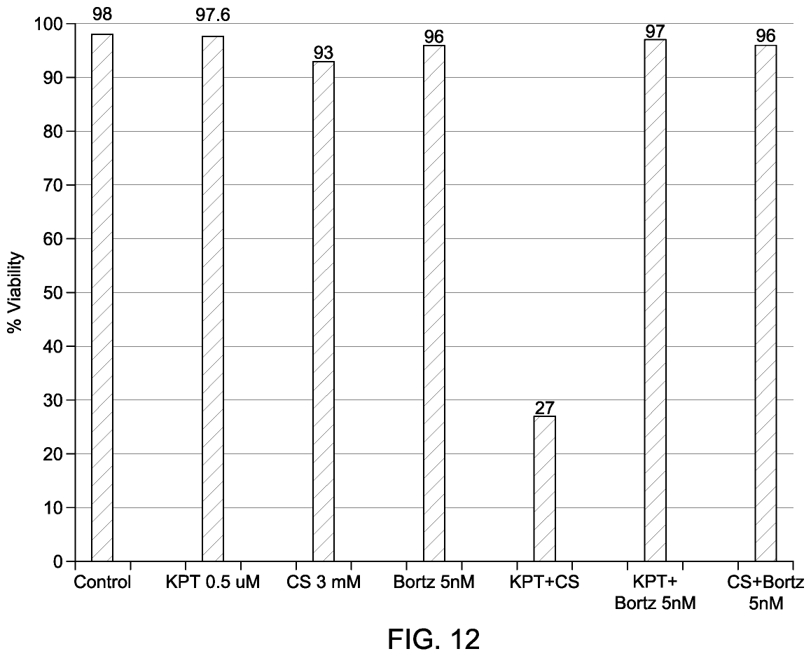

Cell viability of cancer cells treated with aspirin or choline salicylate in combination with a chemotherapeutic was examined. MCL cells or DLBCL cells treated with a combination drug treatment including aspirin at 2.5 mM and gemcitabine at 1 nM showed no change in cell viability (FIG. 11). Similar results were obtained when combining aspirin with bortezomib, when combining choline salicylate with gemcitabine, and when combining choline salicylate with bortezomib. DLBCL cells were treated for 48 hours with a combination drug treatment including selinexor at 0.5 μM, CS at 3 mM, bortezomib at 5 nM, or combinations thereof. Combination treatment with selinexor and CS resulted in decreased cell viability, while all other treatment groups showed now change in cell viability (FIG. 12).

These results demonstrate that the synergy of the combination treatments is specific for the selinexor and salicylate combination.

### Concentrations of Selinexor and of Salicylates

Combination treatments using various concentrations of selinexor, various concentrations of CS, and various treatment times were evaluated. CS concentrations did not exceed 4 mM as higher concentrations are supra-physiologic and would be difficult to achieve in humans.

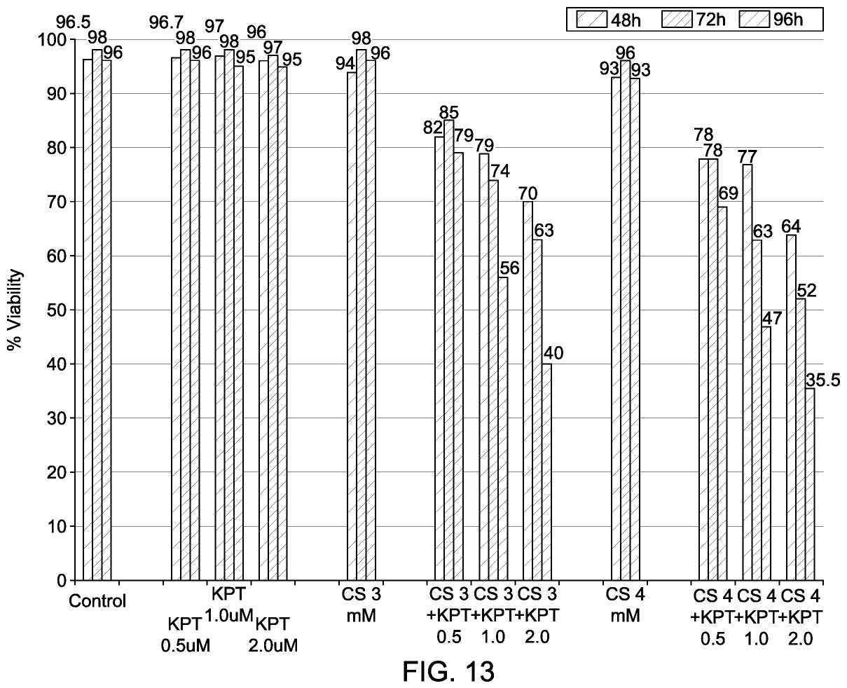

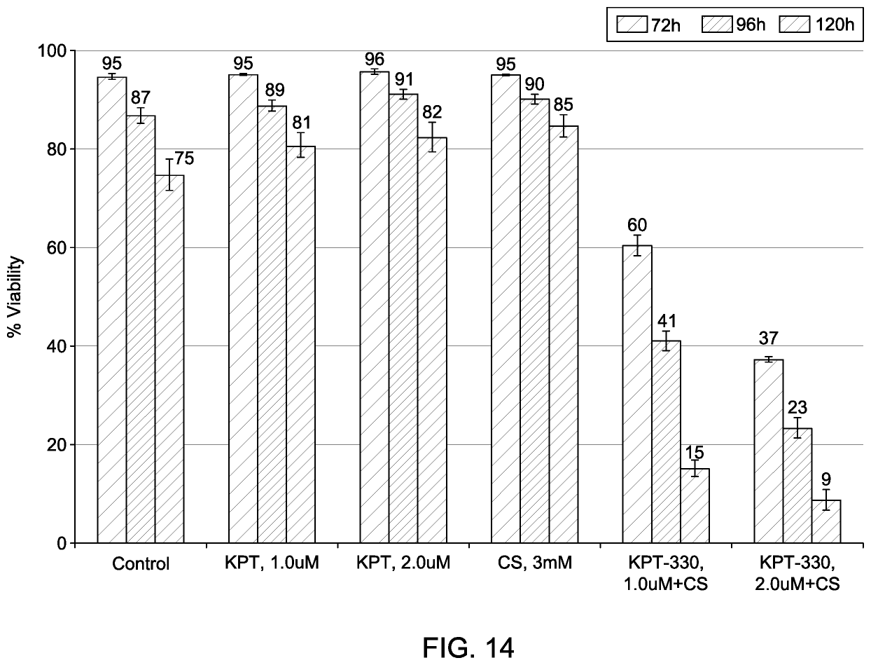

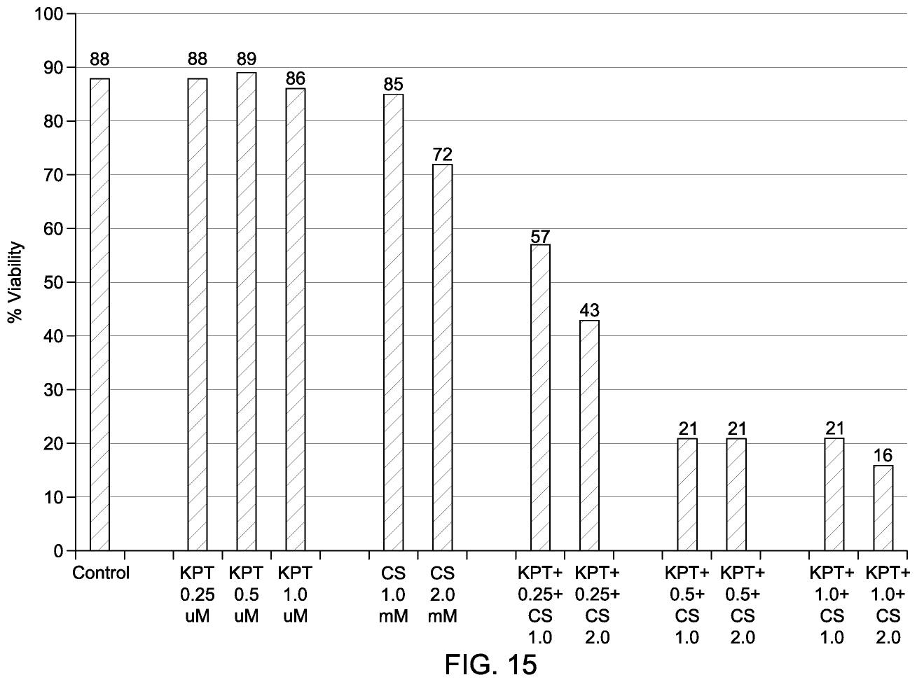

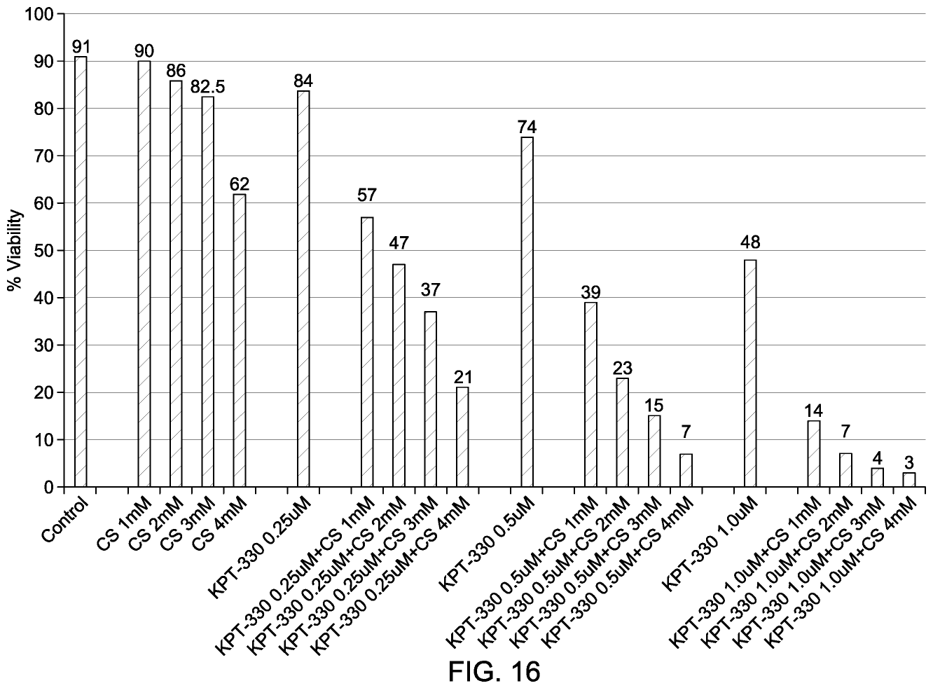

DLBCL cells treated for 48, 72, or 96 hours with a combination drug treatment including selinexor at 0.5 μM, 1.0 μM, or 2.0 μM and CS at 3 mM or 4 mM each showed decreases in cell viability (FIG. 13). DLBCL cells treated for 72, 96, or 120 hours with a combination drug treatment including selinexor at 1.0 μM and CS at 3 mM showed decreases in cell viability (FIG. 14). DLBCL cells treated for 96 hours with a combination drug treatment including selinexor at 0.25 μM, 0.5 μM, or 1.0 μM and CS at 1 mM or 2 mM each showed decreases in cell viability (FIG. 15). MCL cells treated for 72 hours with a combination drug treatment including selinexor at 0.25 μM, 0.5 μM, or 1.0 μM and CS at 1 mM, 2 mM, 3 mL, or 4 mM each showed decreases in cell viability (FIG. 16).

Together these results demonstrate that salicylates (e.g., aspirin or choline salicylate) act synergistically with an inhibitor of CRM1 polypeptides (e.g., Selinexor). Accordingly, one or more salicylates can be used to enhance the antitumor effect of one or more inhibitor(s) of CRM1 polypeptides such that the inhibitor(s) of CRM1 polypeptides can be administered to a mammal (e.g., a human) having cancer (e.g., a hematologic cancer) at lower concentrations thus mitigating adverse effects of inhibitor(s) of CRM1 polypeptides.

### Example 2: Salicylates Enhance Antitumor Activity of CRM1 Inhibitors In Vivo

### Methods

KPT-330 and choline salicylate were used via oral gavage in a mantle cell lymphoma NSG (NOD-scid gamma mouse) mouse model.

Mouse groups: Group 1 (control)=4 female mice; Group 2 (CS)=3 male mice; Group 3 (KPT)=4 male mice; Group 4 (KPT+CS)=4 female mice

Four groups of NSG mice with 3-6 mice per group was treated by oral gavage; groups 1-4: (1) placebo (vehicle; 25% of DMSO, 37.5% polyethylene glycol and 37.5% of distal water) given every day 3 weeks, (2) Selinexor at 15 mg/kg (dissolve in the vehicle) given twice a week for 3 weeks, (3) CS at 500 mg/kg given every day 3 weeks, and (4) Selinexor at 15 mg/kg given twice a week for 3 weeks and CS at 500 mg/kg given every day 3 weeks. Each mouse in the study was injected 5×10⁶ cells of Jeko-1, MCL cells. The primary endpoints were assessing the tumor size via nuclear imaging, tumor histopathology for necrosis/apoptosis and adverse effects (AEs). The AEs were monitored based on systemic signs using Body Condition Scoring.

### Results

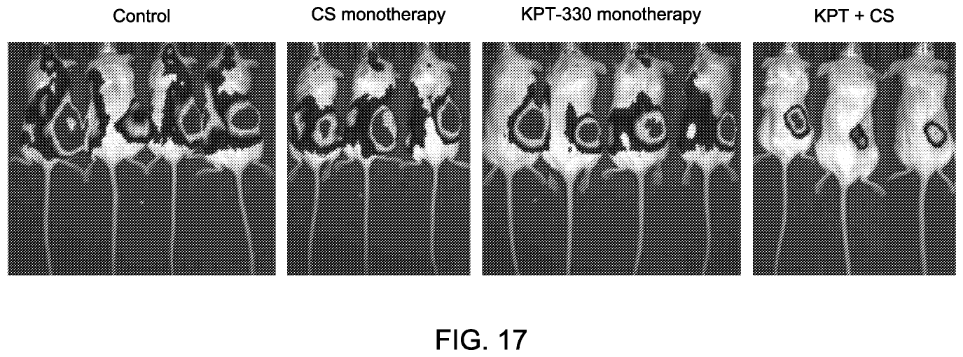

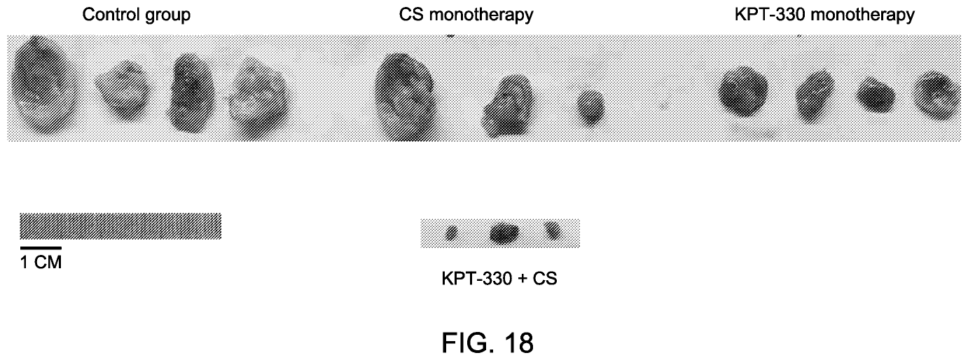

Administering a treatment including selinexor in combination with CS to mice with MCL tumors for 16 days resulted in a reduced tumor size (FIG. 17 and FIG. 18).

These results demonstrate that an inhibitor of CRM1 polypeptides (e.g., selinexor) used in combination with one or more salicylates (e.g., aspirin or choline salicylate) have an antitumor effect, and can be used to a treat a mammal having cancer.

### Example 3: Salicylates Enhance Antitumor Activity of CRM1 Inhibitors In Vivo

This Example demonstrates that salicylate can increase the potency of KPT-330 or other CRM1 inhibitors when used in combination. This combination induces an inhibitory effect on the nuclear export of proteins and inhibits DNA damage repair, DNA synthesis, and cell cycle progression in both hematologic malignancies and solid tumor cells with minimal effects on normal cells. This constellation of anti-tumor effects is unique with respect to other known classes of anti-cancer agents.

### Methods

### Cell Lines

Cell lines were purchased from the cell line repositories ATCC (Manassas, Va.) or DSMZ (Braunschweig, Germany). These included MCL cell lines: JeKo-1, Mino; TCL cell lines: Karpas-299, SR-786; DLBCL cell lines: OCI-Ly1 (LY-1), OCI-Ly3 (LY-3), OCI-Ly19 (LY-19), SU-DHL-6 (DHL-6); MM cell lines: RPMI, U266, OPM2, Xgl, KMS2; ALL cell lines: CRL-1783; non-small cell lung cancer: NCI-H460, A549, HCC827; small cell lung cancer: H1048; sarcoma: Fuji, SW872. Cell lines were cultured according to instruction. Cells used in experiments had viability counts of 90% or greater before treatment.

### Preparation of Primary Patient Samples

Primary patient samples were obtained. The mononuclear cells were obtained from bone marrow, spleen, peripheral blood and lymph nodes as follows. Freshly obtained tissue (lymph node, spleen, bone marrow) was placed in mesh screen and using a syringe plunger, the tissue was pressed into a petri dish. Samples were then rinsed through the screen using measured amounts of RPMI until all tissue has been pressed through. If sample had a visibly large presence of red blood cells, it was processed with ACK Lyse. Subsequently the mononuclear cells were isolated via centrifugation in the presence of ficoll and this step was again repeated following washing the cells with RPMI media.

To isolate peripheral blood mononuclear cells (PBMCs), freshly obtained patient blood sample of patients were centrifuged in the present of ficoll and this step was repeated twice following washing the cells with PBS. Following isolating the mononuclear cells, the viability was assessed and ascertained proper viability above 80% prior to conduct respective experiments. Non-malignant cells were confirmed not to have a malignant condition by pathology review of the tissue.

### Drug Treatment

KPT-330 purchased from Selleckchem (cat no: S7252) was dissolved in DMSO while CS (Santa Cruz, Calif.S 2016-36-6), sodium salicylate (Sigma-Aldrich, cat no: S3007), acetyl salicylate (Sigma-Aldrich, cat no: CAS 50-78-2) was diluted in PBS. Ketorolac (Sigma-Aldrich, cat no: 1356665), Bortezomib (Selleckchem, cat no: S1013), gemcitabine (Sigma-Aldrich, cat no: G6423), Leptomycin-B (Sigma-Aldrich, cat no: L2913-2X) and KPT-185 (Selleckchem, cat no: S7125) were dissolved in DMSO. The concentration range of KPT-330 was 0.05-1.0 μM while CS was 1-3 mM were used for ex vivo treatment and incubation time ranged from 24 hours to 72 hours. For the PARP inhibitor assay, olaparib 10 μM (SelleckChem, cat no: AZD2281 Ku-0059436) was used. Q-VD-OPh (MilliporeSigma, cat no: 551476) was used as the pan-caspase inhibitor to assess caspase induced cell death by K+CS.

### Cell Viability Assessment

The viability assessment for cell lines and primary patients samples were conducted with the Annexin V/PI method. In CMML patient sample, the viability was assessed by counting colony-forming units in respective treatment conditions; KPT-330, CS, K+CS and DMSO control.

### In Vivo Studies, Including Toxicity Assessment

NSG™ (NOD.Cg-Prkdc^{scid}Il2rg^{tm1Wjl}/SzJ) NOD SCID gamma, NOD.Cg-Prkdc^{scid}Il2rgtm1Wjl/SzJ) mice were obtained from in-house breeding colony and 3 million cells of Jeko-1 cells were subcutaneously engrafted in the flank. On day 4 following the inoculation of Jeko-1 cells, KPT-330 at 15 mg/kg, CS 500 mg/kg and the combination were used in respective groups (vehicle, KPT-330 monotherapy, CS monotherapy and K+CS), respectively. Oral gavage was commenced for drug administration and KPT-330 was administered twice a week while CS was administered consecutively 6 days per week. Treatment was commenced for total of 24 days. The vehicle of 20% DMSO, 40% polyethylene glycol and 40% water was used for KPT-330 and water was used to dilute CS to make proper concentrations. The respective week was used in the control groups without the drugs were treatment. Tumor growth was monitored by measuring tumor diameter, tumor within height and the volume was calculated by using length×width (2)/2. Toxicity assessment was done by visual inspection and monitoring weight of the animals during the treatment period. Following autopsy, animal carcasses were fixed in formalin for formal pathology analysis of the internal organs.

### Hematopoietic Progenitor Colony Forming Assay

CMML patient-derived bone marrow samples were first treated with ammonium chloride to deplete red blood cells, washed in RPMI, then plated at a final concentration of 5×10⁴-2×10⁵ cells/mL in addition to drug(s). This solution was immediately inoculated into methylcellulose (StemCell) formulated with recombinant cytokines to support the optimal growth of erythroid progenitor cells, granulocyte-macrophage progenitor cells, and multipotent granulocyte, erythroid, macrophage and megakaryocyte progenitor cells. Plates were incubated with respective treatment conditions at 37° C. and colonies were enumerated on day 10-14.

Synergy Assessment with IC50 and Combination Index (CI)

Jeko-1 and OCI-Ly1 cell lines were used to assess the IC50. Keeping CS at 3 mM, KPT-330 was titrated. Relative IC50 was calculated as described elsewhere (see, e.g., Sebaugh et al., Pharmaceutical statistics 10:128-134 (2011)). The combination index was calculated as described elsewhere (see, e.g., Chou et al., Cancer Res 70:440-446 (2010)).

### Primary Antibodies

CRM1 (Cell Signaling, Danvers, Mass.; catalog number: 46249, dilution 1:1000), Rad51 (abeam; catalog number: ab133534, dilution 1:1000), BublB (Cell Signaling, Danvers, Mass.; catalog number: 4116, dilution 1:1000), cyclin B1 (Cell Signaling, Danvers, Mass.; catalog number: 4138, dilution 1:1000), thymidylate synthase (Cell Signaling, Danvers, Mass.; catalog number: 9045, dilution 1:1000), Aurora A (Cell Signaling, Danvers, Mass.; catalog number: 91590, dilution 1:1000), PLK1 (Cell Signaling, Danvers, Mass.; catalog number: 4513, dilution 1:1000) and mouse anti-human beta-actin antibody (Santa Cruz Biotechnology, Dallas, Tex.; catalog number: SC-47778) were used, and were followed with fluorescent secondary antibodies; anti-rabbit IRDye 800CW or anti-mouse IRDye 700 CW (LI-COR). The membranes were imaged on a LI-COR Odyssey CLX imager.

### Immunoblotting

Cells were extracted in lysis buffer containing protease inhibitor, PMSF, and HALT phosphatase inhibitor for total cellular proteins. The cell lysates were diluted in Laemmli sample buffer supplemented with beta-mercaptoethanol. The proteins were resolved in precast 4-15% gradient, or 7.5% or 10% fixed Criterion TGX Midi protein gels (Bio-Rad) by electrophoresis, and then transferred to PVDF membranes. The membranes were blocked with 1:1 LI-COR ODB/PBS buffer and probed with respective primary antibodies followed with fluorescent secondary antibodies.

### Mass Spectrometry-Based Proteomics Analysis of Cell Lysates

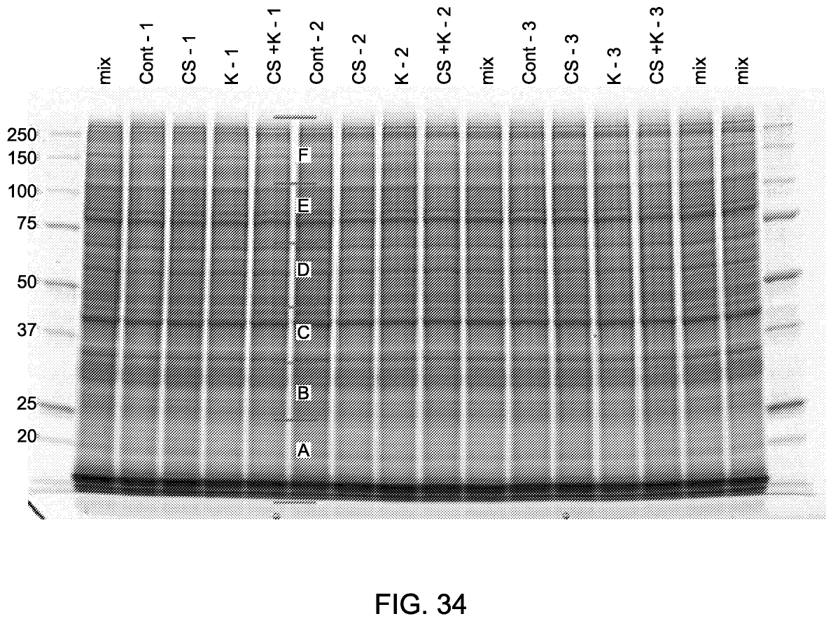

A GeLC-MS/MS method for identifying and quantifying the proteins present in different drug treatment groups was used. Triplicate cell pellets (containing ˜5 million cells/pellet) from each treatment group were individually washed with PBS and solubilized in 2% SDS/100 mM triethylammonium bicarbonate buffer (pH 8). Solubilized pellets were vortexed for 30 seconds, incubated on ice for 5 minutes, and was repeated twice. Proteins were denatured via heating and shaking for 10 minutes at 85° C. Final protein concentrations were estimated using BCA protein assay (Thermo-Fisher, Waltham, Mass.). Protein mixture with 20 μg of was diluted in SDS buffer (5% β-mercaptoethanol), heated for 5 minutes at 85° C., loaded on a 10% Criterion TGX gel (Bio-Rad Labs), and electrophoresed. Sample lanes were divided into six equal horizontal segments for mass spectrometry analysis (FIG. 34) and each gel band was stored in 200 mM Tris at 4° C. for further processing.

Gel bands were destained and dehydrated. Proteins in the bands were reduced with 50 mM TCEP for 50 minutes at 60° C., dehydrated with acetonitrile, and alkylated with 25 mM iodoacetamide/50 mM Tris for 50 minutes at room temperature in the dark. Reduced and alkylated proteins were incubated with 80 μL of 0.002 μg/μL trypsin (Promega, Madison, Wis.) overnight at 37° C. Peptides were extracted by incubating the digests with 20 μL of 4% trifluoroacetic acid and 60 μL of acetonitrile at room temperature for 40 minutes. A second acetonitrile extraction was performed and saved with the initial extraction. All extracts were dried and stored at −20° C.

Dried trypsin digested samples were suspended in 0.2% formic acid/0.1% TFA/0.002% zwittergent. A portion of each digest was analyzed by nano-flow liquid chromatography electrospray tandem mass spectrometry (nanoLC-ESI-MS/MS) using a Q-Exactive HF-X mass spectrometer (Thermo-Fisher Scientific, Bremen, Germany) coupled to a Thermo Ultimate 3000 RSLCnano HPLC system. The digest peptide mixture was loaded onto a 330 nL Halo 2.7 ES-C18 trap (Optimize Technologies, Oregon City, Oreg.), eluted on to 100 μm×33 cm column packed with Agilent Poroshell 120 EC C18 packing (Agilent Technologies, Santa Clara, Calif.). Mass spectrometer analyzed the sample for 90-mins in data dependent MS/MS mode.

### Bioinformatics of Proteomics Data

To analyze the raw data and identify proteins present in the samples and to detect differentially expressed proteins between treatment groups, raw data files were processed using MaxQuant (version 1.6) software configured to use a composite protein sequence database containing Uniprot human reference proteome (downloaded on Mar. 13, 2019) sequences of common contaminants (trypsin, keratin, cotton, wool, etc.). Reversed protein sequences were appended to the database for estimating protein identification false discovery rates (FDRs). The software was configured to use 20 ppm m/z tolerance for precursors and fragments while performing peptide-spectrum matching. MaxQuant inferred semitryptic peptides from the database while looking for the following variable modifications: carbamidomethylation of cysteine, oxidation of methionine and formation of n-terminal pyroglutamic acid. The software filtered peptide and protein identifications at 1% FDR, grouped protein identifications into groups and reported protein group intensities. Protein group intensities were considered as pseudo-quantitative measure of their abundances.

A script written in R programming language performed relative differential expression analysis using protein group intensities. First, protein group intensities of each sample were log 2 transformed and normalized using TMM method. For each protein group, the normalized intensities observed in two groups of samples were modeled using a Gaussian-linked generalized linear model. An ANOVA test was used to detect the differentially expressed protein groups between pairs of experimental groups. Differential expression p-values were FDR corrected using Benjamini-Hochberg procedure. A total of four group comparisons were performed: KPT-330 vs. control, CS vs. control, KPT-330+CS vs. control and CS vs. KPT-330. For each comparison, protein groups with an FDR≤0.05 and an absolute log₂ (fold change)≥2.0 were considered as significantly differentially expressed and saved for further analysis.

### Gene Expression Analysis Using RNA-Seq

To analyze gene expression differences between treatment groups, RNA was extracted using the AllPrep DNA/RNA FFPE kit (Qiagen, Germany). Sequencing libraries were prepared using TruSeq RNA Library Prep kit V2 (Illumina, San Diego, Calif.) and analyzed on a HiSeq 4000 sequencer (Illumina, San Diego, Calif.). Sequenced reads were aligned to the hg38 reference using the MAP-RSeq pipeline slightly modified to use the STAR aligner. Gene-level read counts based on Ensembl version 78 were analyzed using edgeR⁵⁴ to find differentially expressed proteins. For this, gene counts were normalized using TMM method to remove batch effects. Normalized read counts were compared across experimental groups using a negative binomial generalized log-linear model. A total of four group comparisons were performed: KPT-330 vs. control, CS vs. control, K+CS vs. control and CS vs. KPT-330. For each comparison, genes with an adjusted p-value (Benjamini-Hochberg)≤0.05 and an absolute log 2 (fold change)≥2.0 were considered as significantly differentially expressed and saved for further analysis.

### Gene Set Enrichment Analysis (GSEA) and Pathway Analysis

The same methods were utilized for performing GSEA and pathway analysis for both proteomics and gene expression data. For proteomics data, a gene symbol was assigned to each protein group based on the constituent protein identifications. For gene expression data, ensemble transcript ID was mapped to its corresponding gene symbol. From here on, GSEA software (version 4.0) from Broad Institute was used for GSEA analysis. A rank was computed, as −1 log 10(p-value)*sign(fold change), for each gene in each proteomics and gene expression comparison. Genes and their corresponding ranks were utilized for GSEA using GSEAPreRanked method. KEGG, Reactome, HALLMARK and BioCarta gene sets were utilized for enrichment analysis. A custom gene set was derived for NFKB response using public literature and also for CRM1 response genes. Gene sets with a corrected p-value<=0.05 were considered as significant. The Ingenuity Pathway Analysis (IPA, Qiagen, Germany) was utilized for detecting differentially expressed pathways. Only significantly differentially expressed genes/proteins in each comparison were uploaded to IPA. Pathways with an adjusted p-value ≤0.05 were considered for interpretation.

### Immunofluorescence Microscopy

Following 24-48 hours incubation of cells with respective treatment conditions, slides were made through cytospin. Subsequently, slides were fixed with 4% paraformaldehyde and stained with primary antibody. The CRM1 antibody was purchased from Cell Signaling (mAB #46249, dilution 1:1500), gamma-H2AX antibody (Cell Signaling, Catalog number. 80312, dilution 1:1000) were used as primary antibodies and incubated overnight followed by 1 hour incubation of respective secondary antibodies. Subsequently the slides were visualized under confocal microscopy on a Zeiss LSM 780 confocal microscope.

### Comet Assay

Jeko-1 cells and OCI-Ly1 cells were treated with KPT-330, CS, K+CS and DMSO control for 48 hours and comet assay was conducted based on the manufactures protocol (R&D Systems™, catalog no: 4250-050-K). SYBR gold DNA stain was used to stain the DNA under manufactures recommendations. Subsequently focal microscopy was used to image the slides.

### Assessing the Role of Thymidylate Synthase Depletion by Using Thymidine Free Media

Jeko-1 cells were cultured for 5 days with 14365C SAFC EX-CELL® CD CHO Fusion thymidine free media (Millipore Sigma, catalog number: 14365C) in the presence of L-glutamine. After confirming the viability above 90%, cells were treated with the respective drug combinations and single agent treatment with DMSO control for 48 hours in the presence or absence of thymidine (Sigma-Aldrich, cat no: T9250). Subsequently, the viability was assessed via Annexin/PI method as described above.

### Mitotic Index Calculation

Jeko-1 and OCI-Ly1 cells were treated with KPT-330, CS, K+CS and DMSO in respective media. Following the incubation of respective time periods, cytospin slides were prepared and stained with Hema 3 staining on the many factors protocol. Subsequently the mitotic index was calculated by the number of mitoses evaluated in 10 high power fields (×400).

Cell Cycle Analysis Following incubation, cells were fixed with 70% cold ethanol and kept at 4 C° for 24 hours, followed by PI staining. Cell cycle analysis was done by using a BD FACS Caliber flow cytometer (BD Biosciences) and analyzed using FlowJo® Software.

### Double Thymidine Block

A double thymine block was used to synchronize cells at G1 phase of the cell cycle. JeKo-1 cells were cultured in thymidine at 2 mM in RPMI for 9 hours. Subsequently, cells were washed and cultured in RPMI media (RPMI) without additional thymidine for 12 hours and the second block of cell cycle with thymidine (2 mM) was carried out for an additional 9 hours with in RPMI. Following the double thymidine block, cells were released by re-culturing cells in normal RPMI without additional thymidine. The viability of cells at the time of release was >90%. Following the double thymidine block, cells were treated with respective treatment conditions and assess for cell cycle progression.

Assessment of K+CS Activity on Primary Patient Samples with Ovarian Cancer

### Ex Vivo Tumor Culture and Drug Efficacy Study

Since 3D complex multicellular constructs that can self-assemble to recapitulate specific developmental programs, on arrival of fresh ovarian cancer PDX tissue from animal, after removal of debris (i.e., fat and necrotic material), the tumor tissue were cut into 2-4 mm³ pieces and washed with cold sterile phosphate buffered saline (PBS, Life Technologies Inc.), two or three random pieces were snap frozen and stored at −80° C. for DNA isolation, two random pieces were fixed in formalin for histopathological analysis and immunohistochemistry, and the remainder were processed for cryopreservation or isolate single tumor cells for primary 3D culture. Briefly: carefully transfer up to 2 gram fresh tumor tissue into the gentleMACS C Tube (Miltenyi Biotec Cat #130-093-237) containing 5 mL of digestion buffer (MACS human tumor dissociation kit, Miltenyi Biotec, Germany). Tightly close the C Tube and attach it upside down onto the genleMACS Dissociator, run the genleMACS program h_tumor_01. By end of dissociation, add complete culture medium to stop the reaction, centrifuge and re-suspend samples in fresh complete culture medium, and apply the cell suspension to a cell strainer (40 μM) placed on a 50 mL tube, wash cells several times, and re-suspend cell pellet into a final concentration of 10000/well by plating them into a Corning® spheroid microplates ultra-low attachment (ULA) 96 well plates with black/clear round bottom in 90 μl of complete media (CLS4520, Corning Inc., New York, N.Y.) and 24 hours were allowed to form 3D spheroids. All cells were maintained at 37 C°, 5% CO₂ in humidified atmosphere, only validated tumor cells were used further for ex vivo study. The cells were treated with compounds at the indicated concentration for another 120 hours. Cell viability was measured using the RealTime-Glo™ MT Cell Viability Assay (Cat. #G9711, Promega, Madison, Wis.) and GloMax® discover system (Promega, Madison, Wis.). Cell viability was calculated for each concentration as an average of three replicates and normalized to untreated vehicle controls after 120 hours of incubation. Thresholds of assay success were set to include minimum Relative Luminescence Units (RLU) values in controls and the dynamic range between vehicles and blanks.

Drug concentrations used:
CS starts with 5 mM and 1:2 dilution (5 mM/2.5 mM/1.25 mM/0.625 mM/0.3125 mM/0.15625 mM/0.078125 mM)
KPT-330 starts with 5 uM and 1:2 dilution (5 μM/2.5 μM/1.25 μM/0.625 μM/0.3125 μM/0.15625 μM/0.078125 μM)
The combination with same dose and 1:2 dilution

### Targeted Gene Sequencing

Treatment-naïve PDX tumor tissues were crushed using the Cellcrusher Tissue Pulverizer (Cell Crusher Limited, Cork, Ireland) on dry ice. DNA was then extracted using the standard protocol for Qiagen DNeasy Blood and Tissue kit (Cat #69504; Qiagen, Venlo, Netherlands). Extracted DNA was assayed using the BROCA Cancer Risk panel (University of Washington, Seattle, Wash., USA) to detect mutational aspects of DNA repair or its regulation, including ATM, ATR, BARD1, BRCA1, BRCA2, CDK12, CHEK1, PALB2, RAD51C, TP53, and 43 others (as described in Walsh et al., Proc Natl Acad Sci USA 107:12629-12633 (2010)) as escribed elsewhere (see, e.g., AlHilli et al., Gynecologic oncology 143:379-388 (2016)). The assay completely sequences all exons, non-repeating introns, selected promoter regions, and detects large deletions, duplications, and mosaicism. All deleterious mutations were confirmed by Sanger sequencing. Only clear loss of function mutations and missense mutations with experimental evidence of functional consequences was considered deleterious.

Assessment of K+CS Activity on Primary Patient Samples with Glioma

Two GBM patient-derived xenograft (PDX) explanted cell lines, GBM6 and GBM12 from the Mayo GBM PDX National Resource, were propagated in StemPro Neural Stem Cell media supplemented with L-glutamine and penicillin/streptomycin. Cells were plated at 2000 (GBM6) or 500 (GBM12) cells per well in tissue culture-treated, black-walled plates in 50 μl of media and incubated overnight before treatment with experimental compounds. Cells were treated at respective concentrations of KPT-330 and CS as single agents or in combination. Experiments were incubated for 14 days before viability was analyzed by Cell Titer GLO 3D according to the manufacturer's instructions.

### Nuclear Export Efficiency Assay

A GFP reporter construct was made using the backbone of pEGFP-N1 vector. Specifically, the green fluorescence (GFP) was in-frame tagged with 3 consecutive copies of nuclear localization sequence (NLS) of SV40 large T antigen to the N-terminus and 3 consecutive copies of nuclear export sequences (NES) of HIV Rev to the C-terminus. The resulting construct encodes NLS-GFP-NES fusion protein capable of shuttling freely between the nucleus and the cytoplasm. The construct DNA was transfection into U2OS cells using Lipofectamine 2000 reagent (Life Technologies) according to product instruction. Six hours post transfection, the cells were treated with vehicle, CS, KPT-330, and K+CS for 24 hours. Finally, the treated cells were fixed with 4% PFA, and permeabilized, and stained for endogenous expression of CRM1 protein with anti-CRM1 antibody (Cell Signaling, Danvers, Mass.; catalog number: 46249, dilution 1:1500). Cell images were collected on a Zeiss LSM 780 confocal microscope.

### CRM1-YFP Fusion Expression

Construct pCMV-hCRM1-YFP encoding human CRM1-YFP fusion protein was as described elsewhere (Rodriguez et al., The Journal of biological chemistry 275:38589-38596 (2000)). Hela, U2OS, and HEK293 cells were transfected with the pCMV-hCRM1-YFP construct using Lipofectamine 2000 reagent for 6 hours followed by the treatment of vehicle, CS, KPT-330, and K+CS for 24 hours. Cells were then harvested for Western blotting analysis by probing CRM1 expression with anti-CRM1 antibody (Cell Signaling, Danvers, Mass.; catalog number: 46249, dilution 1:1000). The antibody detects both endogenous CRM1 protein of 120 kDa and CRM1-YFP protein of 145 kDa.

### Statistical Analysis

Matched pair analysis and student's t-test was used to compare continuous variables. A p-value of <0.05 was considered statistically significant, and all analyses were performed using JMP 14.0 software (SAS Institute, Cary, N.C.). Combination index of <1 was defined as synergistic and the CI was calculated by using CalcuSyn software.

### Results

Increased Potency of CRM1 Inhibitors when Combined with Salicylates

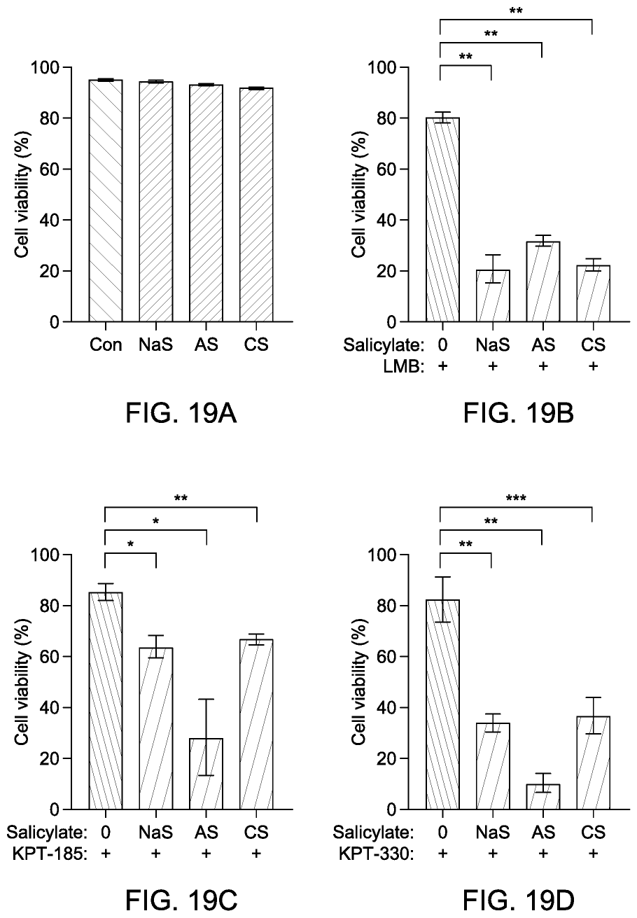

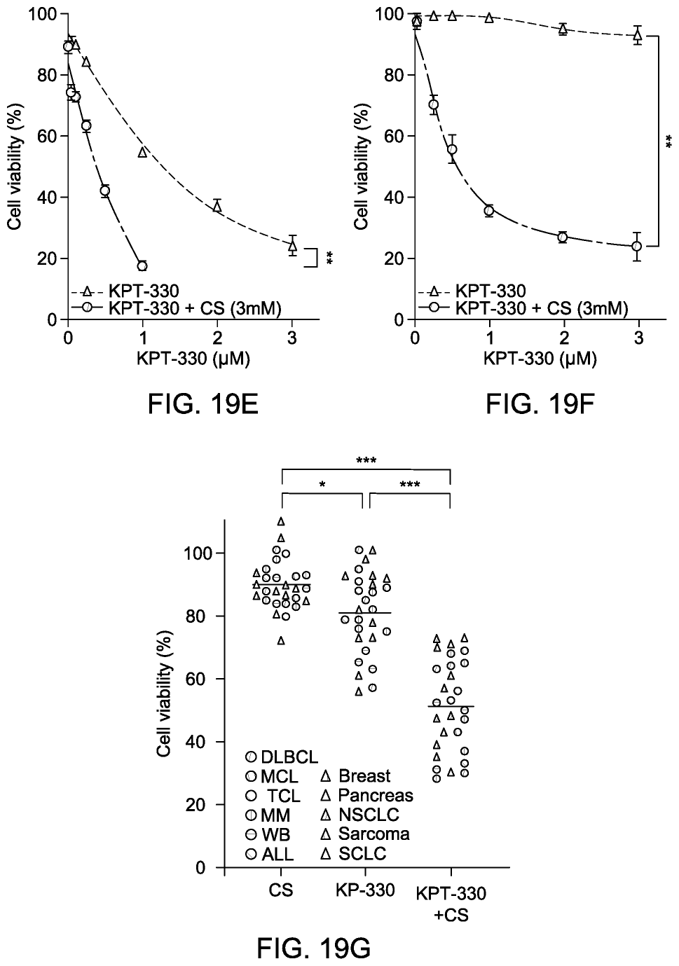

To evaluate if salicylates could potentiate the antitumor effect of CRM1 inhibitors, the antitumor activity of various CRM1 inhibitors; leptomycin B (LMB), KPT-185, and KPT-330 in combination with well-established salicylate compounds; acetyl salicylate (AS), sodium salicylate (NaS) and CS was assessed. Salicylates alone had no effect on cell viability, while CRM1 inhibitors as single-agents had minimal cytotoxicity at low concentrations (FIG. 19a-d). Strong antitumor activity was observed when CRM1 inhibitors at these same lower concentrations were combined with any salicylate compared to CRM1 inhibitors alone (FIG. 19a-d). No synergistic or additive antitumor effect was observed when combining salicylates with traditional chemotherapeutic agents (gemcitabine or bortezomib) or when non-salicylate non-steroidal anti-inflammatory drugs (NSAIDs, ketorolac) were combined with CRM1 inhibitors, suggesting that the synergy between CRM1 inhibitors and salicylates is unique and specific. For further studies, KPT-330 was used as the CRM1 inhibitor, and CS was used as the salicylate. The selected dose range of 1-3 mM of CS that we used in ex vivo experiments is equivalent to achievable and tolerable dose range in humans (see, e.g., Stark et al., Molecular and cellular biology 25:5985-6004 (2005); and Wolf et al., International record of medicine 173:234-241 (1960)). Serial concentrations of KPT-330 were used that included doses less than 1 μM which induce minimal anti-tumor effect in NHL, to 2.5 μM, a concentration approximately equivalent to the approved dose of 80 mg twice a week known to induce responses but with toxicity (see, e.g., Chari et al., New England Journal of Medicine 381:727-738 (2019); Kuruvilla et al., Blood 129:3175-3183 (2017); and Abdul Razak et al., Journal of clinical oncology 34:4142-4150 (2016)). The K+CS decreased the IC₅₀ from 1.3 μM to 0.3 μM and 1.8 μM to 0.4 μM in JeKo-1 (mantle cell lymphoma cell line) and OCI-Ly1 (DLBCL cell line) cells, respectively (FIG. 19e-f). Moreover, KPT-330 concentrations as low as 0.1 μM and 0.25 μM were also synergistic [combination index (CI)<1] with 3 mM CS in both JeKo-1 and OCI-Ly-1 cells, respectively (Table 2). The potent antitumor effect with K+CS treatment was also observed across a broad range of cell lines in hematologic malignancies and solid organ tumors, highlighting the potential broad applicability of the K+CS combination in cancer therapy (FIG. 19g and Table 3). This potency was also seen in RS4-11 cells (CRL-1873) carrying an E571K mutation in the XPO1 gene (cancer.sanger.ac.uk/cell_lines/sample/overview?id=909703; and Tate et al., Nucleic Acids Research 47:D941-D947 (2018)), (Table 3).

TABLE 2
Assessing synergy through Combination Index (CI) in
JeKo-1 and OCI-Lyl cells when treated with K + CS.

| JeKo-1 | | OCI-Lyl | |
|---|---|---|---|
| KPT-330 (μM) | CI at CS 3 mM | KPT-330 (μM) | CI at CS 3 mM |
| 0.005 | 1.02 | 0.25 | 0.01 |
| 0.1 | 0.99 | 0.5 | 0.007 |
| 0.25 | 0.9 | 1.0 | 0.002 |
| 0.5 | 0.72 | 2.0 | 0.001 |
| 1.0 | 0.54 | 3.0 | 0.0009 |

Note:
JeKo-1 cells and OCI-Lyl cells were treated with KPT-330 and CS as single agent or in combination and viability was assessed by Annexin V/PI assay. The combination index (CI) was calculated by using CalcuSyn software and CI of <1 considered to be synergistic.

TABLE 3
Ex vivo effect of K + CS in hematologic malignancies and solid tumors.

| | | | | | P-values | | |
|---|---|---|---|---|---|---|---|
| | | % viability normalized for | | | Control | KPT-330 | CS |
| | | respective controls | | | vs. | vs. | vs. |
| Disease type | Cell line | KPT-330 | CS | K + CS | K + CS | K + CS | K + CS |

Hematologic malignancies

| | | | | | | | |
|---|---|---|---|---|---|---|---|
| MCL | JeKo-1 | 95 | 93 | 43 | <0.0001 | 0.0002 | <0.0001 |
| | Mino | 89 | 86 | 89 | | | |
| TCL | SR-786 | 91 | 100 | 56 | <0.0001 | <0.0001 | <0.0001 |
| | Karpas-299 | 88 | 80 | 53 | | | |
| DLBCL | OCI-Ly1 | 101 | 101 | 64 | <0.0001 | 0.03 | <0.0001 |
| | OCI-Ly3 | 85 | 84 | 63 | | | |
| | OCI-Ly19 | 57 | 88 | 30 | | | |
| | DHL6 | 96 | 101 | 80 | | | |
| MM | RPMI | 87.5 | 85 | 68 | <0.0001 | <0.0001 | <0.0001 |
| | U266 | 75 | 93 | 37 | | | |
| | OPM2 | 82 | 98 | 65 | | | |
| | Xg1 | 76 | 95 | 28 | | | |
| | KMS2 | 79 | 83 | 47 | | | |
| WM | BCWM | 69 | 84 | 31 | 0.01 | 0.12 | 0.01 |
| | MWCL | 65 | 89 | 50 | | | |
| | RPCI | 63 | 92 | 52 | | | |
| ALL | CRL-1873 | 79 | 91.5 | 33 | 0.001 | 0.001 | <0.0001 |

Solid tumors

| | | | | | | | |
|---|---|---|---|---|---|---|---|
| Pancreatic | Panc-1 | 90 | 90 | 71 | 0.001 | 0.01 | 0.008 |
| cancer | L3.6 | 61 | 72 | 30 | | | |
| Non-small cell | H460 | 93 | 105 | 47 | 0.003 | 0.002 | 0.016 |
| lung cancer | A549 | 101 | 94 | 73 | | | |
| | HCC827 | 78 | 90 | 48 | | | |
| Small-cell | H1048 | 81 | 92 | 73 | | | |
| lung cancer | | | | | | | |
| Sarcoma | FuJi | 93 | 85 | 70 | 0.003 | 0.001 | 0.0004 |
| | SW872 | 82 | 88 | 61 | | | |
| Breast cancer | Hs 578T | 73 | 87 | 43 | 0.0013 | 0.0048 | 0.01 |
| | BT-474 | 73 | 110 | 39 | | | |
| | BT-20 | 58 | 89 | 35 | | | |
| | MCF7 | 98 | 87 | 57 | | | |

Note:
Respective cell lines were treated with KPT-330 concentration ranging from 0.1 μM-1.0 μM, and CS concentration ranging from 1 mM-3 mM as single agent or in combination for 48 hours. The concentrations of KPT-330 and CS that gave the best synergy were selected and maintained constant through the treatment conditions of a given sample. Cell viability was assessed by Annexin V/PI assay. Potent antitumor effect was observed when KPT-330 was combined with CS. MCL: Mantle cell lymphoma; TCL: T-cell lymphoma; DLBCL: Diffuse large B-cell lymphoma; MM: Multiple myeloma; WM: Waldenstrom macroglobulinemia; ALL: Acute lymphoblastic leukemia; CS: choline salicylate; K + CS: KPT-330 + CS.

### K+CS Treatment is Efficacious In Vivo

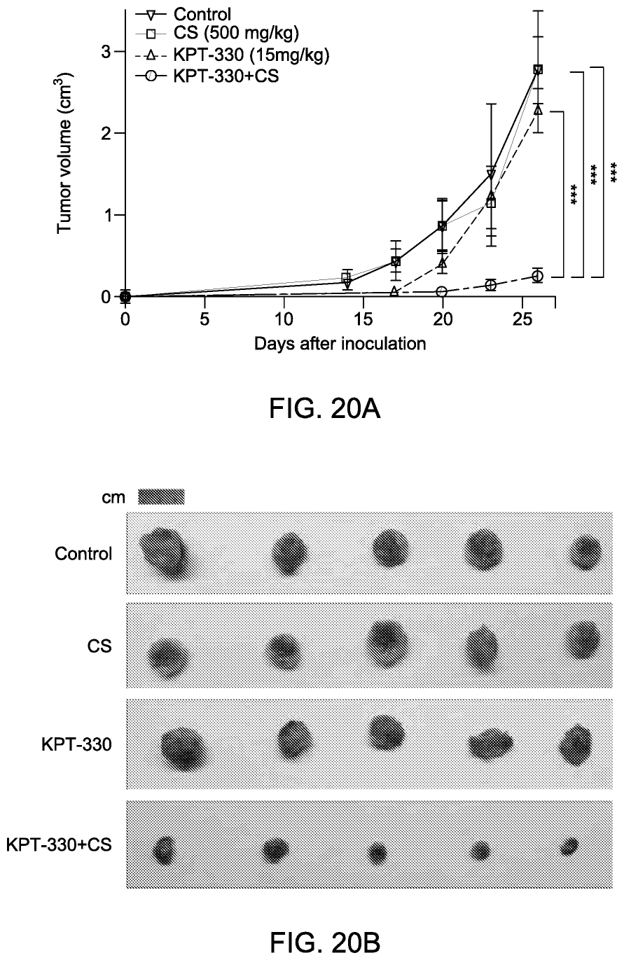

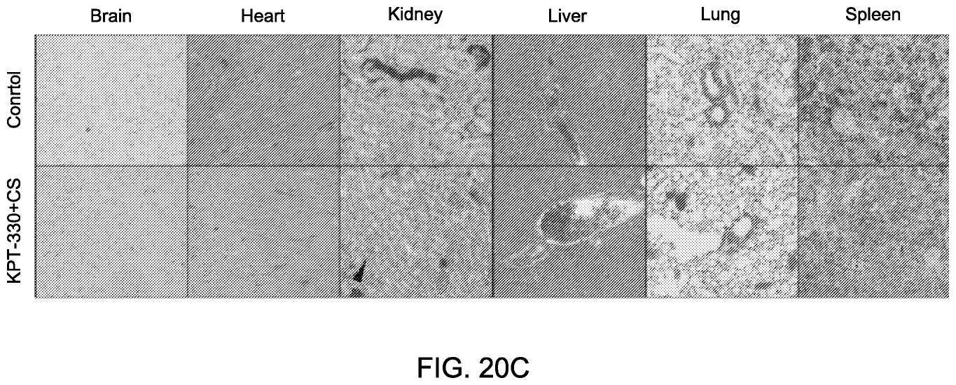

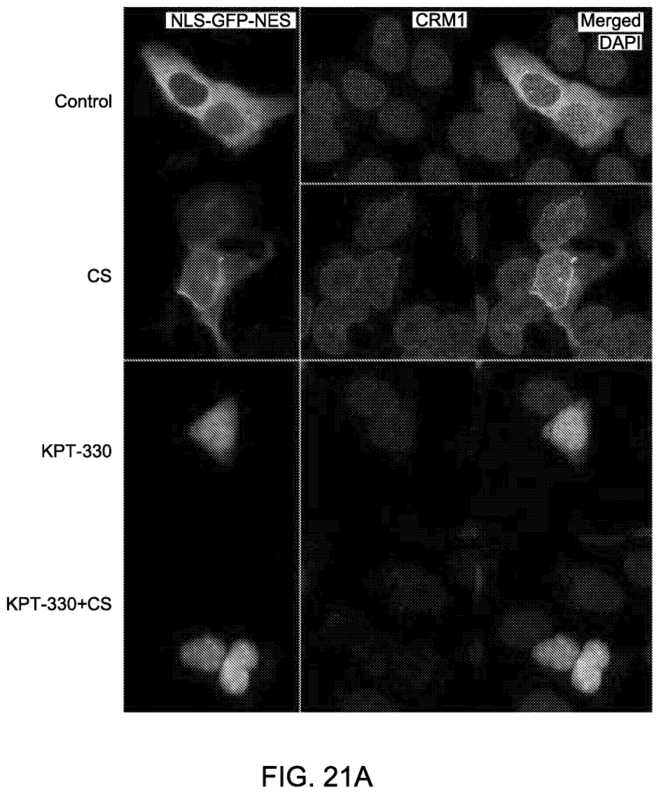

Given the ex vivo efficacy of K+CS in various cell lines, K+CS was tested on tumor xenografts in NSG (NOD.Cg-Prkdc_{scid} Il2rg^{tm1Wjl}/SzJ) mice subcutaneously engrafted with JeKo-1 cells. Tumor-bearing mice were randomized to treatment with vehicle, low dose KPT-330 (15 mg/kg) or CS (500 mg/kg) alone, or K+CS combination at respective concentrations administrated by oral gavage. Validating our ex vivo results, significant decrease in tumor volume and growth rate were demonstrated in the K+CS group compared to KPT-330 or CS treated groups as single agents or vehicle treated controls in vivo (FIG. 20a-b). Moreover, the K+CS combination was well tolerated without apparent AEs such as weight loss or treatment-related mortality in tumor bearing mice.

### K+CS Treatment is not Toxic to Normal Organs in Mice

To assess AEs of K+CS on normal organs, a toxicology assessment was performed in non-tumor-bearing NSG mice treated with K+CS or vehicle alone. Mice were observed daily for toxicity and autopsied on day 26. The K+CS treatment did not induce any treatment-related morbidity or mortality, and no significant toxicities such as weight loss were evident. No visceral toxicity was noted following independent pathology analysis of the brain, lungs, heart, liver, kidneys, and spleen tissues. Grade I renal tubular hyperplasia was observed in 1/5 control mice and 4/5 mice in the treatment group (FIG. 20c).

K+CS is a Better Inhibitor of Nuclear Export than KPT-330 Alone

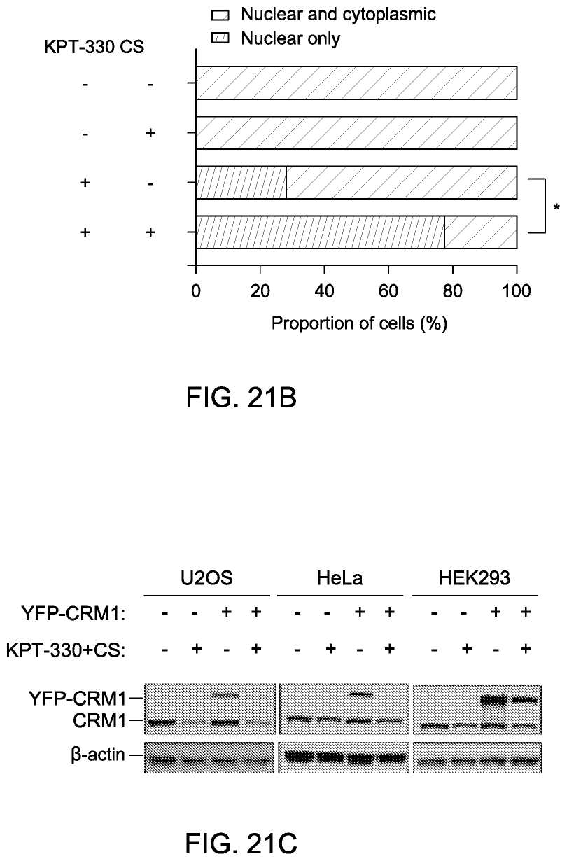

The findings of robust antitumor activity of K+CS combination both ex vivo and in vivo led us to explore the mechanism(s) of this effect. Since KPT-330 is a CRM1 inhibitor, the efficiency of the nuclear export and the spatial expression of CRM1 protein in K+CS treated cells were first examined. To assess nuclear export function, an engineered reporter construct encoding the green fluorescent protein (GFP) carrying the nuclear localized sequence (NLS) and the nuclear export sequence (NES) was transiently transfected into U2OS, a sarcoma cell line in which the nuclear and cytoplasmic compartments are easily visualized. In untreated cells, the reporter protein freely shuttled between the nucleus and cytoplasm (FIG. 21a, control). With K+CS treatment, complete nuclear localization of the GFP was observed compared to an incomplete nuclear localization in cells treated with KPT-330 alone at 0.5 μM (FIG. 21a). To quantify the efficiency of nuclear export, 100 reporter-expressing cells were scored for complete versus incomplete nuclear localization of GFP. As shown in FIG. 21b, 78% of cells in the K+CS group had complete nuclear localization of GFP compared to 28% of cells treated with KPT-330 alone, (p=0.02). Decreased expression of CRM1 protein was also observed by immunofluorescence in K+CS treated cells compared to CS or KPT-330 treated cells as single agents or DMSO controls (FIG. 21a). Taken together, K+CS treatment leads to more efficient inhibition of nuclear export than KPT-330 alone, and such inhibition is associated with a significant reduction of nuclear CRM1 protein expression.

### K+CS Treatment Enhanced the Degradation of CRM1 Protein

To further investigate the mechanism of the reduction of CRM1 protein expression in K+CS treated cells, U2OS, HeLa, and HEK293 cells were treated with K+CS for 24 hours followed by immunoblotting for CRM1 protein. Endogenous CRM1 protein expression was decreased with K+CS treatment compared to control. To determine if this was due to suppression of XPO1 (gene encodes CRM1 protein) expression at the transcription level, or increased degradation of CRM1 protein at the protein level, a construct expressing YFP-CRM1 fusion (YFP-CRM1) under a non-native CMV promoter was transfected into U2OS, HeLa, and HEK293 cells and treated with K+CS for 24 hours. It was observed that K+CS treatment reduced the level of CRM1-YFP fusion protein similar to the endogenous CRM1 protein in all cell lines (FIG. 21c). Since different promoters drive the transcription of endogenous CRM1 and the CRM1-YFP transgene, the finding that both these proteins were similarly reduced suggests post-translational protein degradation is occurring.

### K+CS Uniquely Affects Cellular Proteins Involved in Cell Cycle, DNA Damage Repair, and DNA Synthesis

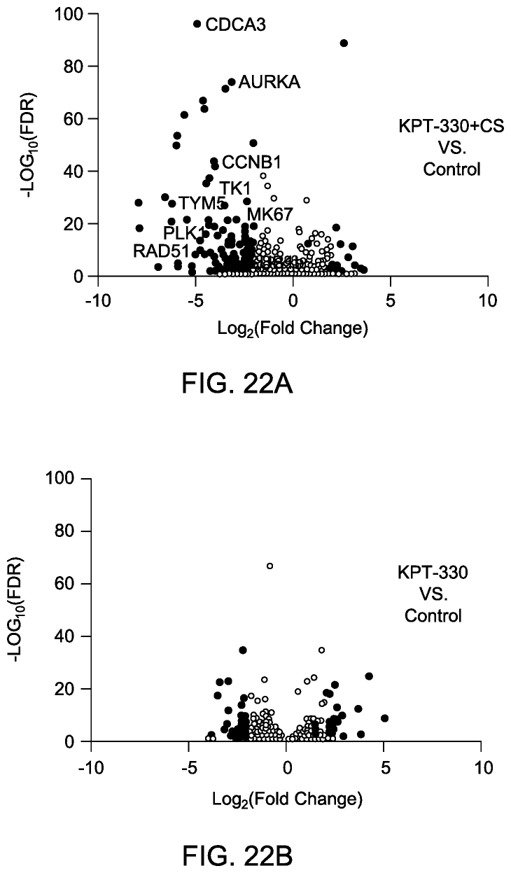

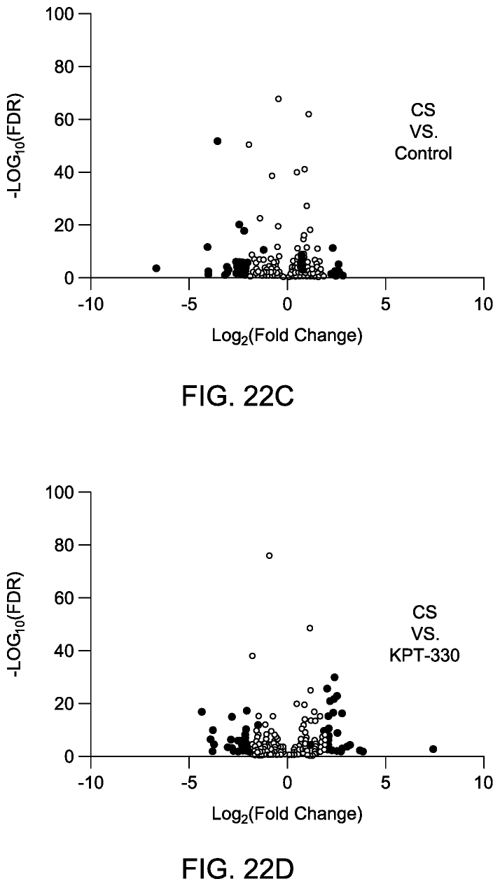

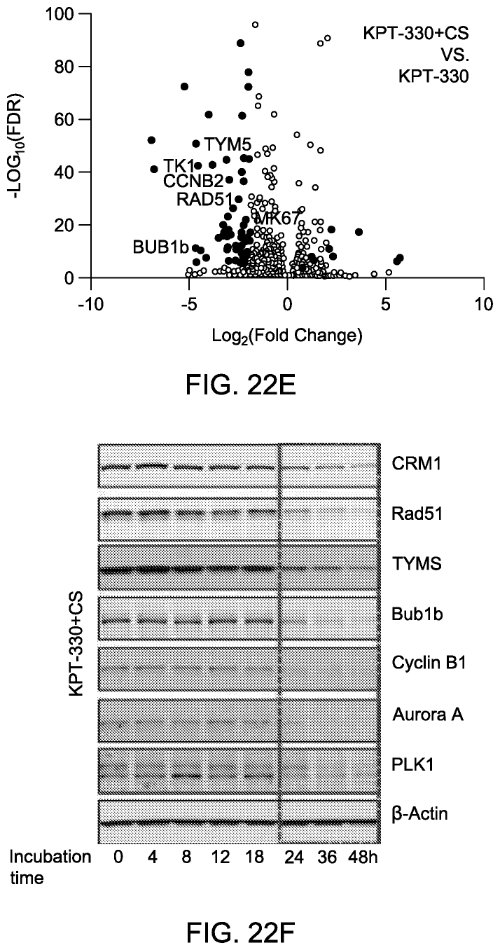

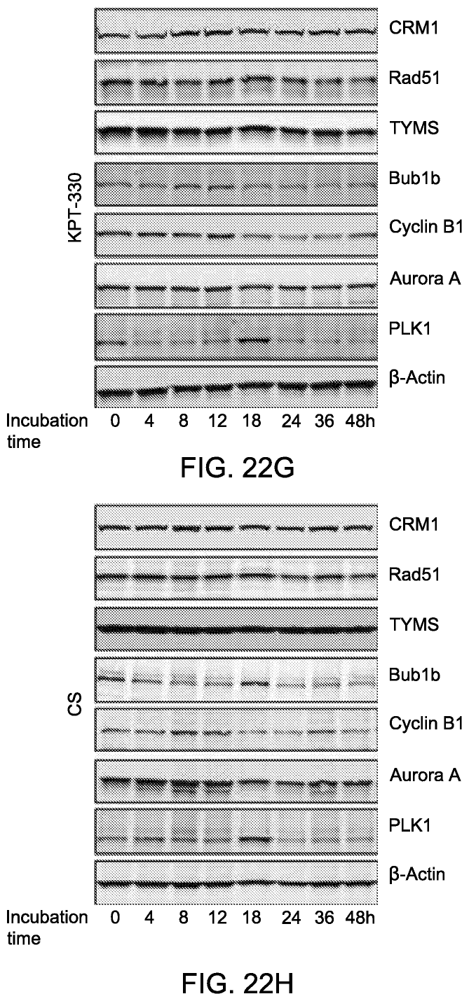

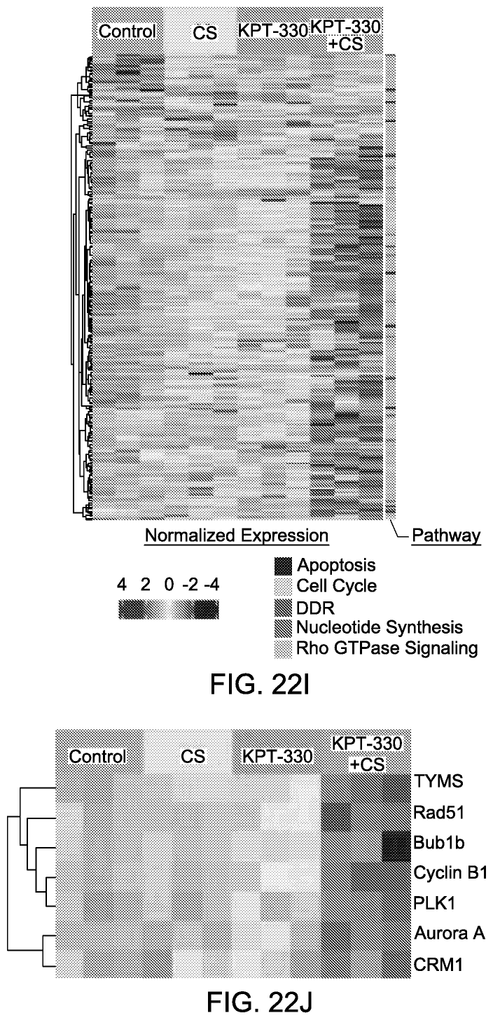

The decreased expression of CRM1 protein supported further experiments to determine if other proteins were affected by K+CS. Proteomic analysis by mass spectroscopy (MS) was performed to catalog protein expression changes occurring in JeKo-1 cells with K+CS treatment. A group of about 100 proteins was identified where the expression was uniquely affected by K+CS treatment including Rad51, thymidylate synthase (TYMS), Bub1b, polo-like kinase 1 (PLK1), aurora kinase A (AURKA), and Cyclin B1 (CCNB1) (FIG. 22a-e, 4j). These results were validated by immunoblotting in JeKo-1 (FIG. 22f-h) as well as U2OS, OCI-Ly1, HeLa, and HEK293 cells. Furthermore, to understand if the downregulation is specific to certain biological processes, Gene Set Enrichment Analyses (GSEA) was conducted, and it was found that the affected proteins are associated with DNA synthesis, DNA damage repair, and mitotic checkpoint pathways (FIG. 22i).

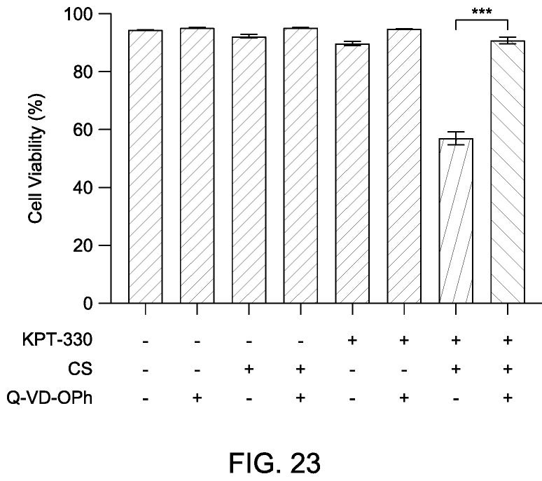

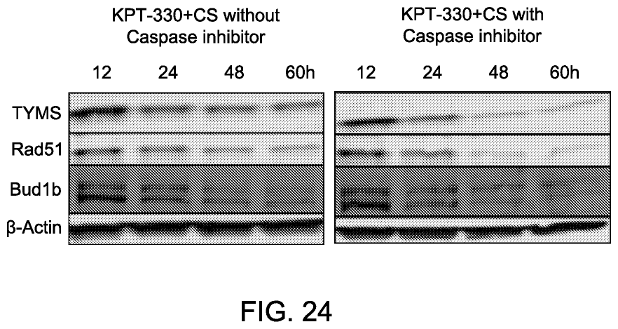

To exclude the possibility that the decreased expression of these proteins was mediated by caspases during apoptosis, the role of caspase-induced cell death in the expression of proteins involved in the aforementioned pathways was tested by using a pan-caspase inhibitor, Q-VD-OPh. The presence of Q-VD-OPh rescued cells from K+CS induced cell death (FIG. 23). However, adding Q-VD-OPh to K+CS treatment did not prevent the decreased expression of the proteins tested (FIG. 24). These results suggest that K+CS induces caspase mediated programmed cell death¹⁸ through affecting the expression of these proteins.

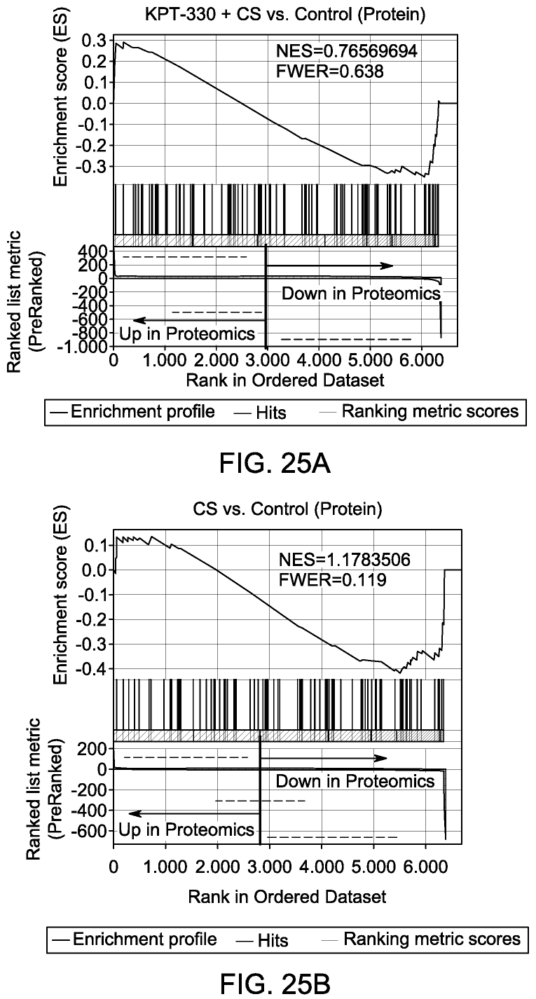

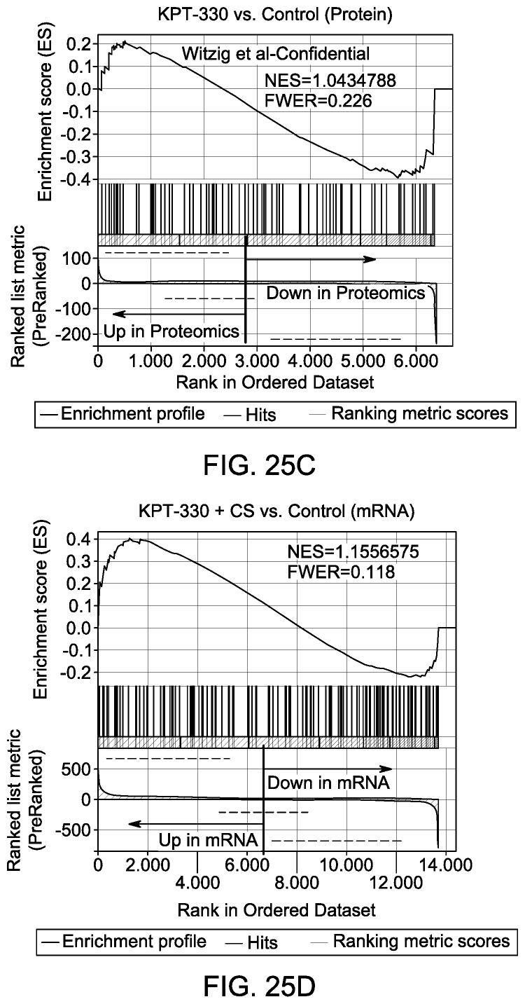

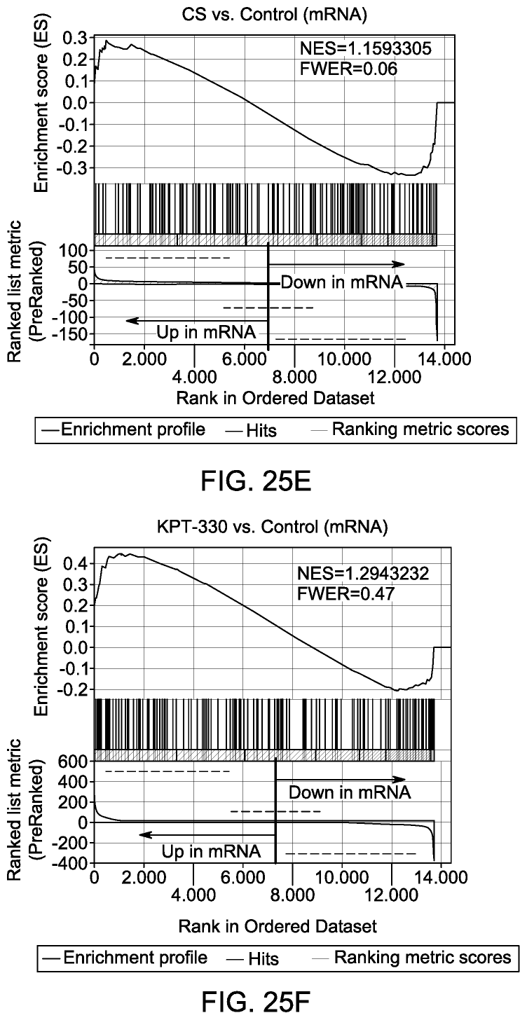

Data from proteomic analysis was also utilized to test our original hypothesis that K+CS would affect the NFkB signaling pathway. The Ingenuity Pathways Analyses and GSEA determined that the expression of NFkB signaling pathway proteins were not significantly altered (FIG. 25).

### Cell Cycle Analysis

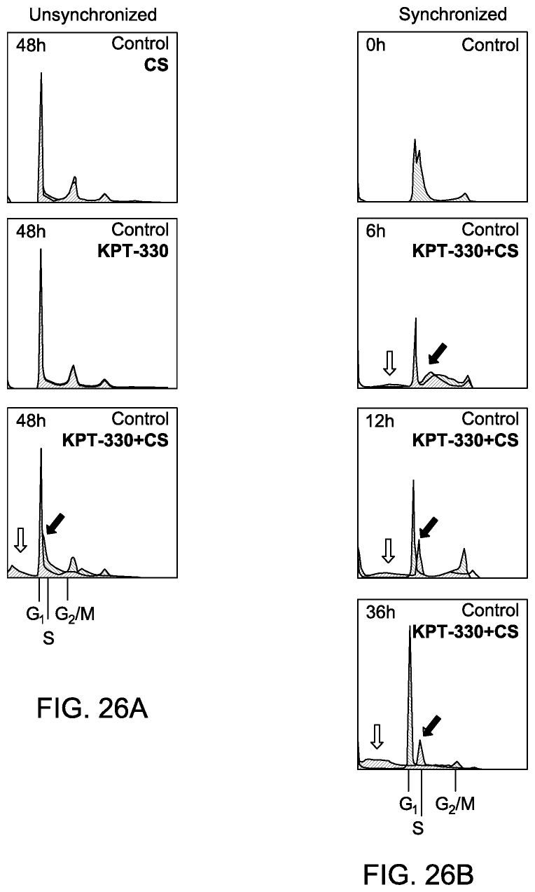

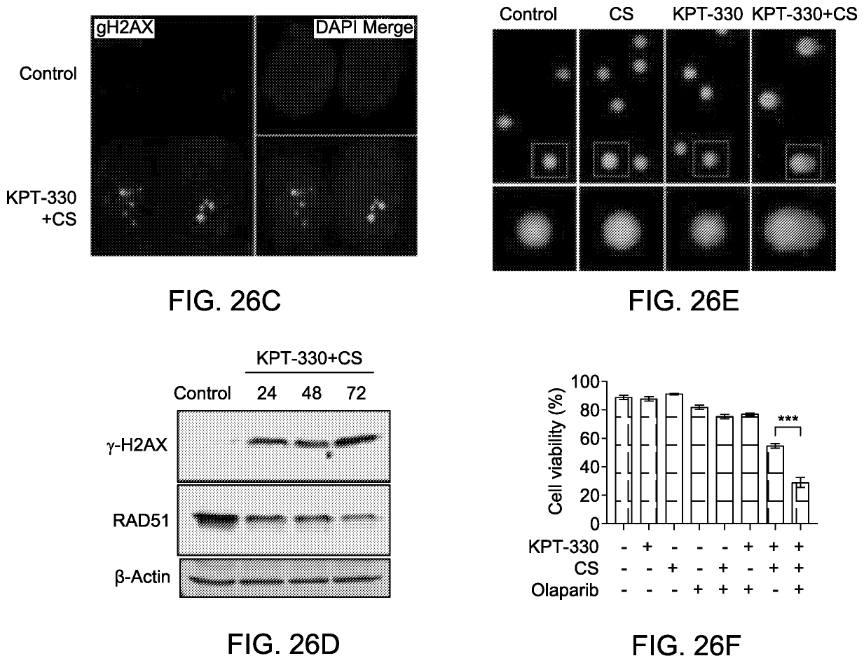

Whether K+CS treatment may affect cell cycle progression, DNA damage repair and DNA synthesis was evaluated. It was found that K+CS treatment of JeKo-1 cells uniquely blocked the cell cycle in S-phase and induced apoptosis compared to cells treated with KPT-330 or CS alone or controls. The fraction of cells in the G2/M phase was 12%, 15%, 11% and 0.7% in cells treated with DMSO control, KPT-330, CS or K+CS, respectively (FIG. 26a). To validate these findings, JeKo-1 cells were synchronized with a double thymidine block followed by release in the presence of K+CS treatment and assessed the cell cycle at various timepoints. It was observed S-phase blockade coinciding with the emergence of the apoptotic cell population (FIG. 26b). Given the S-phase arrest, the decreased percent of G2/M-phase was independently validated through calculating the mitotic index (MI) in JeKo-1 cells by light microscopy. The MI was 5/10 high power fields (HPF) after 48 hours of K+CS treatment compared to 166/10 HPF, 153/10 HPF and 166/10 HPF in CS only, KPT-330 only treated cells and controls, respectively (p=0.0005 for K+CS vs KPT-330). These data suggest that K+CS blocks S-phase progression and prevents cells from entering the G2/M phases of the cell cycle.

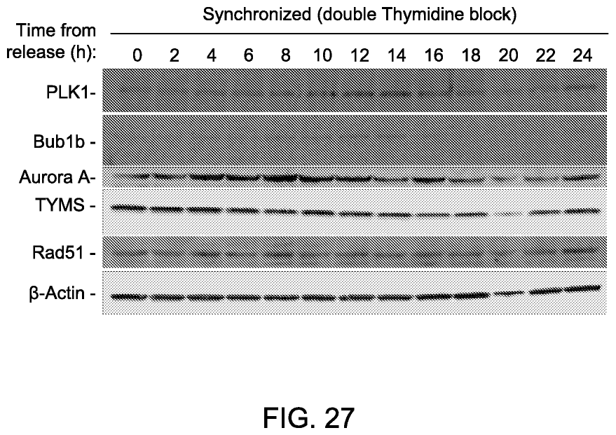

To determine whether some proteins specific to stages outside of S-phase should be under-expressed, especially those specific for G2/M-phase, a protein expression database (cyclebase.org/CyclebaseSearch) was used. More than one third of the downregulated proteins were specific for G2/M including Bub1, Bub1b, AURKA, CDCA3, and PLK1. To validate this, the expression of proteins specific for S-phase (Rad51 and TYMS) or G2/M-phase (AURKA, Bub1b, and PLK1) was profiled by immunoblotting. In untreated JeKo-1 cells, AURKA, Bub1b, and PLK1 were only expressed in G2/M whereas Rad51 and TYMS were expressed throughout the cell cycle (FIG. 27).

### Evaluating for DNA Damage Following K+CS Treatment

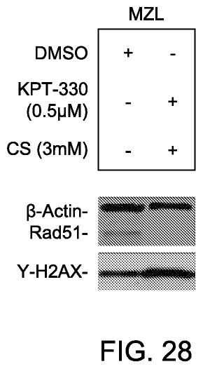

To investigate the anti-tumor mechanism(s) of K+CS, Rad51 and TYMS were evaluated. In view of the findings that K+CS induced cell cycle arrest in S-phase and caused decreased expression of Rad51 (FIGS. 22a, 22f, and 22j), it was evaluated whether K+CS was working through a DNA damage repair pathway. If so, the reduced level of Rad51 would compromise DNA repair resulting in the persistence of unrepaired DNA breaks in K+CS treated cells and the accumulation of serine 139 phosphorylated H2AX (γ-H2AX) at DNA repair foci, the hallmark of ongoing DNA damage repair. As shown in FIG. 26c, strong y-H2AXpositive foci were readily detectable by immunofluorescence in K+CS treated JeKo-1 cells but not in control. In a second independent approach, strong expression of γ-H2AX protein was observed by immunoblotting with concomitant decrease of Rad51 in K+CS treated JeKo-1 cells (FIG. 26d) and a primary patient sample with marginal zone lymphoma (FIG. 28) that was not observed in the control cells.

To further demonstrate that these γ-H2AX positive DNA foci are the evidence of unproductive DNA repair due to the reduced Rad51 expression, a Comet assay that directly detects the integrity of cellular DNA was performed. K+CS-treated JeKo-1 exhibited DNA fragmentation resulting in a “comet-like” DNA mobility profiles (FIG. 26e). This observation further confirmed that K+CS treated cells harbor a significant level of un-repaired DNA breaks, a condition known to be detrimental to cell cycle progression and survival.

### PARP Inhibitors Further Potentiate the Antitumor Effect of K+CS

The reduced expression of Rad51 with K+CS treatment offers an opportunity to further enhance anti-tumor activity through Rad51 insufficiency. Since Rad51 and BRCA proteins are essential for DNA homologous recombination (HR), and BRCA deficiency can lead to synthetic lethality in malignant cells treated with Poly (ADP-ribose) polymerase (PARP) inhibitors, whether K+CS treated cells would also be sensitive to PARP inhibitors such as olaparib was evaluated. Olaparib alone or with single-agent KPT-330 or CS did not have any antitumor activity (FIG. 26f). However, K+CS with olaparib induced more cytotoxicity than K+CS alone, suggesting K+CS treatment induces a phenotype equivalent to BRCA deficiency. These results not only functionally confirm the role of K+CS in the downregulation of Rad51 protein, but also create a new opportunity for potential combinations with olaparib.

### The Combination Treatment Depresses DNA Synthesis by Affecting Thymidine Synthesis

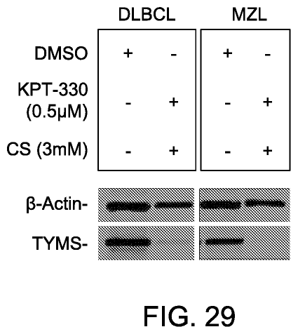

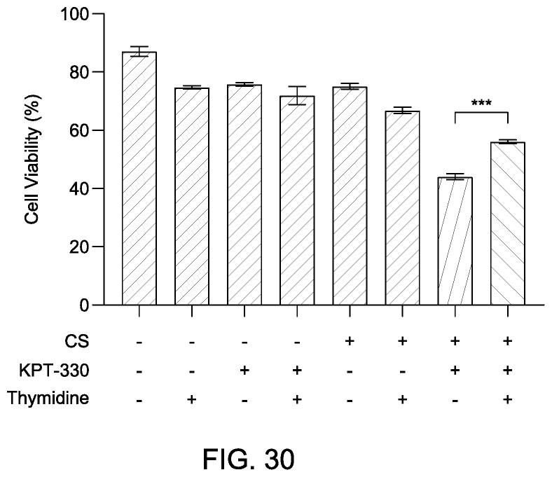

Given the decrease of TYMS protein expression in cell lines (FIG. 22g), and primary patient samples with K+CS (FIG. 29), the role of TYMS in K+CS-induced antitumor activity was explored. Cells were cultured in thymidine-free media and treated with KPT-330 and CS alone or in combination with or without thymidine. The addition of exogenous thymidine produced a statistically significant increase in cell viability after K+CS treatment (FIG. 30) suggesting that the K+CS antitumor effect also involves, at least in part, the TYMS-mediated pyrimidine synthesis pathway.

### The Effect K+CS Treatment on the Cellular Transcriptome

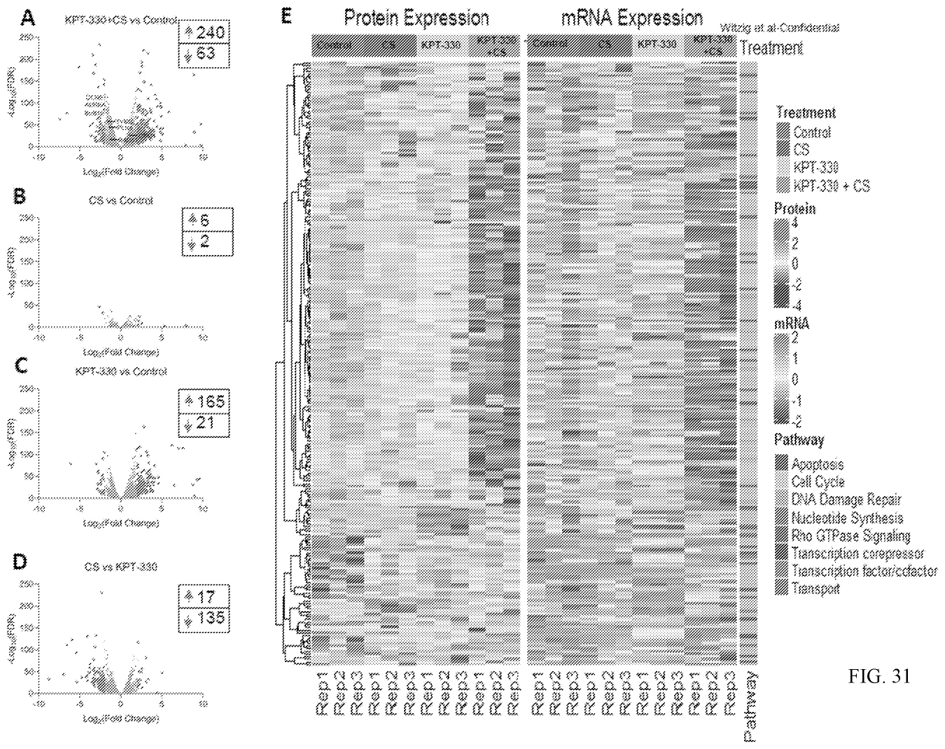

Given that gene transcription requires efficient nucleocytoplasmic transport of many regulatory proteins, the effect of K+CS treatment on global or pathway-specific gene expression was investigated. The analysis of the cellular transcriptome by RNA sequencing showed that the transcripts of the respective proteins involved in DNA damage repair, DNA synthesis and cell cycle arrest were uniquely down-regulated with K+CS treatment (FIG. 31a). Of interest, the transcription of CRM1 was actually upregulated by K+CS treatment, further confirming that protein degradation as a major mechanism behind the diminution of CRM1 expression (FIG. 31a). Moreover, the pathway analysis of the affected transcriptome aligned with that of the effected proteins, thereby confirming that K+CS treatment uniquely effects the transcription of proteins involved in DNA damage repair, DNA synthesis and cell cycle progression (FIG. 31e; Table 4). This result is also consistent with the notion that some of the affected proteins are merely secondary to S-phase arrest.

TABLE 4
Comparison of pathway analysis conducted by using proteomic and transcriptomic data.

| | | | | | Gene Expression |
|---|---|---|---|---|---|
| | | | Proteomics | | (RNA-Seq) |
| | | | Pathway | | Pathway |
| Pathway Category | Ingenuity Canonical Pathways | P-value | Impression | P-value | Impression |
| Apopotosis | Apoptosis Signalling | 0.0135 | Upregulated | 0.0170 | Upregulated |
| Cell Cycle | Cell Cycle Control of Chromosomal Replication | 0.0156 | NA | 0.0110 | NA |
| Cell Cycle | Cell Cycle: G2/M DNA Damage Checkpoint Regulation | 0.0000 | Downregulated | 0.0003 | Downregulated |
| Cell Cycle | Cycling and Cell Cycle Regulation | 0.0055 | Downregulated | 0.0001 | Downregulated |
| Cell Cycle | Mitotic Roles of Polo-Like Kinase | 0.0003 | Downregulated | 0.0000 | Downregulated |
| Cell Cycle | p53 Signaling | 0.0100 | Upregulated | 0.0174 | Upregulated |
| Cell Cycle | Role of CHK Proteins in Cell Cycle Checkpoint Control | 0.0036 | Upregulated | 0.0115 | Upregulated |
| DNA Damage Response | Role of BRCA1 in DNA Damage Response | 0.0000 | Downregulated | 0.0359 | Downregulated |
| DNA Damage Response | DNA Damage-induced 14-3-3g Signaling | 0.0306 | NA | 0.0029 | NA |
| Nucleotide Synthesis | Pyrimidine Deoxyribonucleotides De Novo Biosynthesis I | 0.0028 | Downregulated | 0.0452 | NA |
| Nucleotide Synthesis | Pyrimidine Ribonucleotides De Novo Biosynthesis | 0.0324 | NA | | |
| Nucleotide Synthesis | Pyrimidine Ribonucleotides Interconversion | 0.0282 | NA | | |
| Nucleotide Synthesis | Salvage Pathways or Pyrimidine Ribonucleotides | 0.0427 | Downregulated | | |
| Nucleotide Synthesis | tRNA Charging | 0.0191 | Downregulated | | |
| Nucleotide Synthesis | 3-Phosphoinotiside Biosynthesis | | | 0.0442 | Downregulated |
| Other | April Mediated Signaling | 0.0191 | Downregulated | | |
| Other | Aryl Hydrocarbon Receptor Signaling | 0.0045 | Downregulated | | |
| Other | ATM Signaling | 0.0141 | Downregulated | 0.0177 | Downregulated |
| Other | B Cell Activating Factor Signaling | 0.0224 | Downregulated | | |
| Other | Death Receptor Signaling | 0.0032 | Downregulated | | |
| Other | EIF2 Signaling | 0.0079 | Downregulated | | |
| Other | Estrogen-mediated S-phase Entry | 0.0005 | Downregulated | 0.0004 | Downregulated |
| Other | NER Pathway | 0.0016 | Downregulated | | |
| Other | PPARα/RKRα Activation | 0.0105 | Upregulated | | |
| Other | Role of JAK1, JAK2 and TYK2 in Interferon Signaling | 0.0033 | NA | | |
| Other | TNFR1 Signaling | 0.0427 | Downregulated | 0.0425 | NA |

Note:
Differentially expressed proteins and genes between KPT-330 + CS vs. control were subjected to pathway analysis using Ingenuity Pathway Analysis (IPA) software. Pathways that were significantly enriched (corrected p-value <=0.05 and number of proteins or genes changed in the pathway >=3) were shown here. Pathway impression summarizes the expected direction of pathway change, which is generated as a Z-score by IPA.

### K+CS has Potent Antitumor Effects on Primary Patient Samples

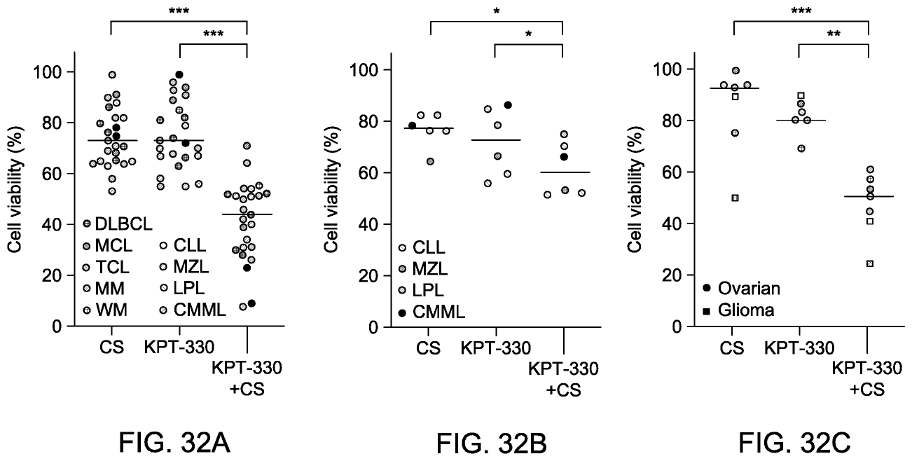

Strong antitumor effects were observed with K+CS compared to single agents or DMSO control in fresh primary patient tumor samples ex vivo (FIG. 32a). More importantly, the antitumor effect was even more potent in aggressive hematologic malignancies such as transformed DLBCL, DLBCL with MYC translocation and BCL2 BCL6 rearrangement, MM with high risk cytogenetics, ibrutinib-resistant mantle cell lymphoma, high-risk chronic lymphocytic leukemia (CLL) resistant to ibrutinib, high-risk CLL with TP53 deletion refractory to ibrutinib and idelalisib based regimens (Table 5). To assess the effects of K+CS on nonmalignant human cells, mononuclear cells from peripheral blood, spleens, lymph nodes and bone marrows obtained from patients without a histopathological or flow cytometry proven diagnosis of malignancy were tested and minimal cytotoxicity was found compared to malignant cells (FIG. 32b; Table 5). These observations suggest potent cytotoxicity in high proliferative tumors and less effect on normal cells for K+CS.

TABLE 5
Assessment of the antitumor effect by K + CS on primary patient samples.

| | | % viability normalized | | | P-values | | |
|---|---|---|---|---|---|---|---|
| | | for respective controls | | | Control | KPT-330 | CS |
| | | KPT- | | | vs. | vs. | vs. |
| Disease | Patient samples | 330 | CS | K + CS | K + CS | K + CS | K + CS |
| DLBCL | NOS | 63 | 76 | 30 | 0.0003 | 0.0004 | 0.002 |
| | Double hit transformed from FL | 82 | 71 | 52 | | | |
| | Double expresser transformed | 68 | 65 | 28 | | | |
| | from CLL/SLL | | | | | | |
| | Double expresser | 74 | 80 | 52 | | | |
| | Double hit, replaced from RCHOP, | 81 | 86 | 44 | | | |
| | ASCT and BR | | | | | | |
| Indolent | Relapsed splenic MZL | 55 | 63 | 34 | <0.0001 | <0.0001 | <0.0001 |
| lymphoma/ | Relapsed splenic MZL | 67 | 71 | 51 | | | |
| leukemia | Relapssd splenic MZL | 70 | 82 | 51 | | | |
| | Relapsed splenic MZL | 67 | 65 | 40 | | | |
| | Relapsed nodal MZL | 58 | 65 | 31 | | | |
| | Lymphoplasmacytic lymphoma with IgG | 79 | 88 | 54 | | | |
| | monoclonal gammopathy | | | | | | |
| | WM | 85 | 79 | 55 | | | |
| | CMML* | 56 | 53 | 8 | | | |
| | Relapsed MCL | 89 | 68 | 39 | | | |
| | Relapsed MCL resistant to Ibrutinib | 94 | 91 | 71 | | | |
| | Relapsed CLL: Ibrutinib resistent, TP53 deleted | 99 | 75 | 23 | | | |
| | Relapsed CLL: Ibrutinib and Idalalisib resistant, | 72 | 78 | 9 | | | |
| | TP53 deleted | | | | | | |
| T-cell | Relapsed T-cell Lymphoblastic leukemia | 70 | 82 | 31 | 0.0096 | 0.04 | 0.05 |
| lymphoma/ | Relapsed Peripheral T-cell lymphoma | 73 | 64 | 42 | | | |
| leukemia | Relapsed Peripheral T-cell lymphoma | 68 | 73 | 51 | | | |
| MM/plasma | Relapsed with high risk cytogenetics | 79 | 58 | 46 | 0.0011 | 0.0005 | 0.01 |
| cell leukemia | Relapsed with high risk cytogenetics | 91 | 90 | 64 | | | |
| | Relapsed with high risk cytogenetics | 93 | 69 | 54 | | | |
| | Kappa light chain | 55 | 64 | 26 | | | |
| | myeloma/plasma cell leukemia with I(11; 14), | | | | | | |
| | standard risk | | | | | | |
| | Relapsed with high risk cytogenetics | 96 | 99 | 50 | | | |
| Nonmalignant | Splenic MNC | 84 | 82 | 70 | 0.0002 | 0.01 | 0.003 |
| cells | Splenic MNC | 66 | 64 | 53 | | | |
| | Splenic MNC | 59 | 76 | 51 | | | |
| | Splenic MNC | 58 | 76 | 52 | | | |
| | BM MNC | 78 | 82 | 74 | | | |
| | PBMC | 86 | 78 | 66 | | | |

Note:
Cells from primary patient samples were obtained freshly from respective tissue sources. Subsequently, cells were treated with KPT-330 (0.05-0.5 μM) and CS (1-3 mM) as single agent or in combination. Viability assessment was performed at 48 h with Annexin V/PI assay. The respective concentrations of KPT-330 and CS were chosen at the range where best synergistic antitumor effect was observed and maintained constant through the treatment conditions of a given sample. DLBCL: diffuse large B-cell lymphoma; CLL: chronic lymphocytic leukemia; SLL: small lymphocytic lymphoma; WM: Waldenstrom macroglobulinemia; CMML: chronic myelomonocytic leukemia; R/R: relapsed and/or refractory; MNC: mononuclear cells; BM: bone marrow; PBMC: peripheral blood mononuclear cells; RCHOP: rituximab, cyclophosphamide doxorubicin, vincristine, and prednisone; ASCT: autologous stem cell transplant; BR: bendamustine rituximab;
*the antitumor effect was assessed by counting colony forming units in respective conditions as the cells were cultured in methyl cellulose media.

### The Combination Drug Treatment is Effective in Solid Tumors

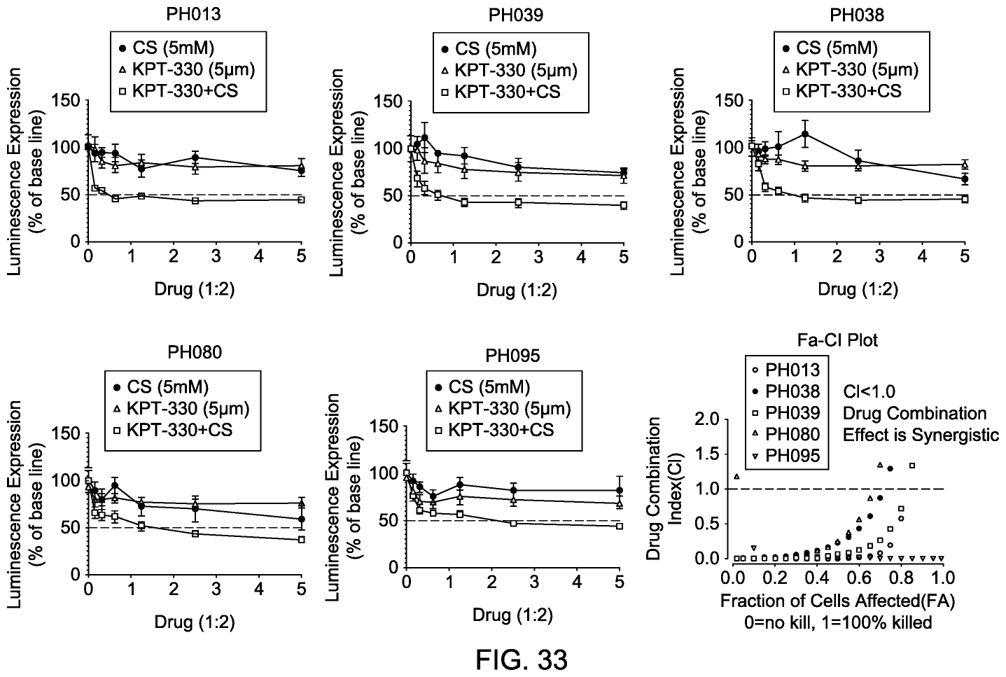

Having demonstrated the antitumor activity of K+CS on hematologic malignancies and cell lines derived from solid tumors (FIG. 19g), ovarian cancer tissue samples obtained from patient derived xenograft (PDX) models were tested. Eleven ovarian cancer samples were treated ex vivo with K+CS, KPT-330 or CS as single agents or with DMSO control. In three of the 11 samples, single agent KPT-330 induced a significant antitumor effect, making the assessment of synergy impossible. In the other eight samples, five (63%) showed significant synergistic antitumor effects unique to K+CS with a combination index <1.0 at 50% fraction affected (FIG. 32c; FIG. 33). Of these five patients, three had known mutations or under-expression in BRCA, Rad51c or CDK12 genes. One patient lacked mutations in BRCA1/2 but had a phenotype for homologous recombination deficiency (disease-free >9 years from completion of frontline treatment) and one patient had no available clinical outcome or sequencing data. Three of the 8 (37%) were resistant to K+CS treatment, and were HR proficient.

Since both KPT-330 and salicylates penetrate the blood-brain barrier, the activity of K+CS was also assessed in gliomas. Two PDX tumor samples-one aggressive fibrillary astrocytoma and one glioblastoma-were tested ex vivo and potent cytotoxicity with K+CS treatment was observed (FIG. 32c).

These results demonstrate that a KPT-330 and choline salicylate (CS) combination (K+CS) has the potential to change cancer treatment for many tumor types.

### Example 4: Selinexor Enhance Antiviral Activity of CRM1 Inhibitors

To examine the effect of selinexor on viral propagation SARS-CoV2 infected eukaryotic cells, selinexor was administered at concentrations ranging from 10 ηM to 3 μM in two settings: (a) prophylactic (pre-treatment) of cells with selinexor prior to infection and (b) concurrent treatment with selinexor at the time of infection (co-incubation).

For a prophylactic treatment, Vero (immortalized monkey cells) cells were incubated for 6 hours with selinexor at concentrations of 0, 10, 30 and 100 nM prior to viral infection for 1 hour at 37° C. and incubated for 4 days. Following this, viral titers were assessed in a standard plaque assay. For a concurrent treatment, Vero cells were infected for 1 hour at 37° C. in the presence of selinexor (0-100 nM). Cells were then incubated until analysis for 4 days.

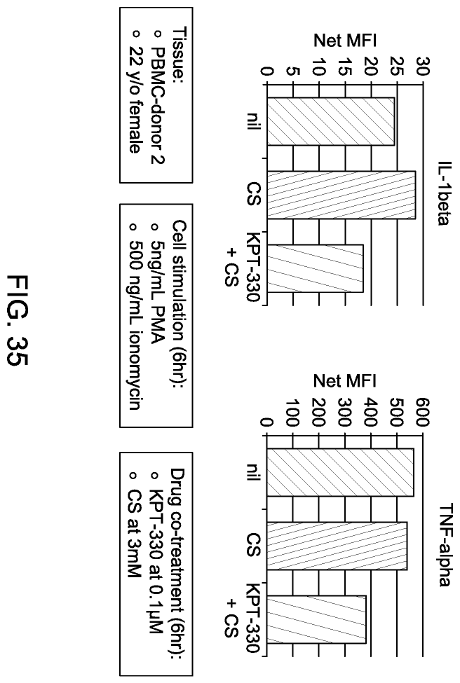

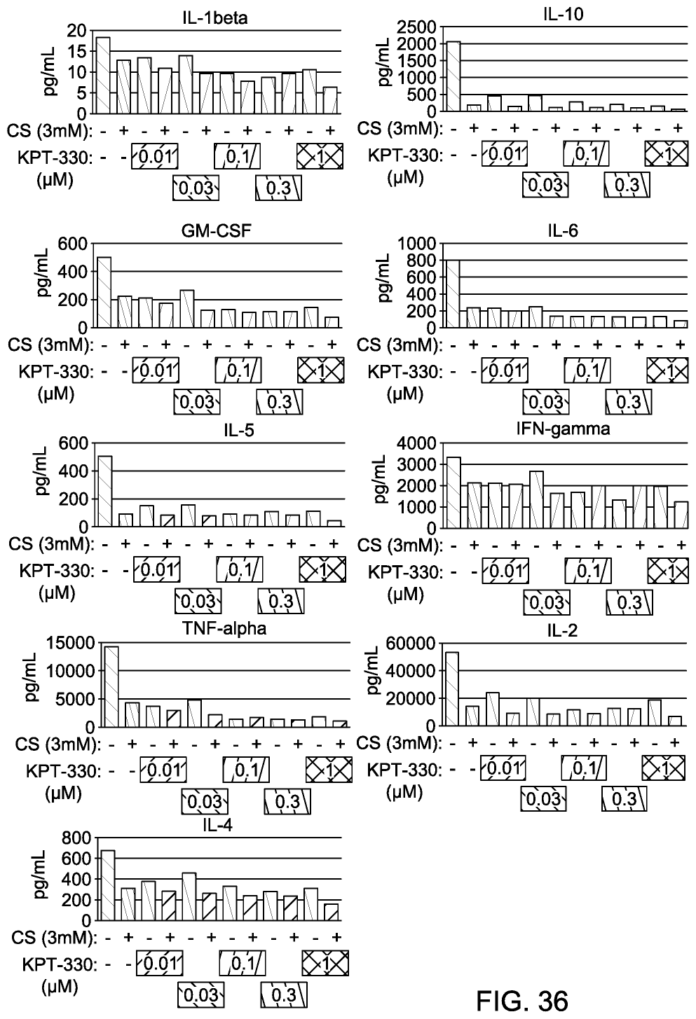

To evaluate the immune response, cytokines were measured in cells that were treated in vitro with Selinexor+/−CS. In some experiments, tissues were obtained from a 22-year-old female, and were stimulated for 6 hours (37° C.) with 5 ng/mL PMA and 500 μg/mL ionomycin. In other experiments, cells (2×10⁶ PBMCs) were obtained from a 61-year-old male, and were stimulated for 6 hours (37° C.) with 500 ng/mL PMA and 10 μg/mL ionomycin. Tissues or cells were then treated with KPT-300 for 6 hours (37° C.) in the presence or absence of CS (3 mM). Co-treatment with a combination of KPT-330 and CS reduced cytokine expression in cells (FIGS. 35 and 36).

The combination of selinexor with CS inhibited production of inflammatory cytokines that lead to the development of acute respiratory distress syndrome (ARDS), such as IL-1, GMCSF, TNF-alpha and IL6.

These results demonstrate that the combination of KPT-330+CS can be used to exhibit anti-inflammatory properties, and can thus be used to treat inflammation. In some cases, the combination of KPT-330+CS can be used to treat one or more diseases that are driven by and/or associated with a pro inflammatory state.

### Example 5: KPT-330 and CS as a Treatment for COVID-19

Using KPT-330 and CS together (K+CS) as a treatment for COVID-19, inhibits viral replication and decreases cytokine production, thereby simultaneously decreasing the infectivity as well as the pro-inflammatory response.

The SARS-Cov2 nucleocapsid protein is overexpressed in human cells using cDNA constructs, and the infected cells are treated with K+CS to evaluate whether the nucleocapsid protein is localized to the nucleus.

Monkey Vero E6 cells are infected with SARS-Cov2, and the infected are treated with K+CS to evaluate whether co-treatment inhibits the reproduction of the SARS-Cov2 virus.

Two cohorts of patients who are either 1) patients with mild symptoms (cohortl: outpatients), or 2) hospital patients with severe symptoms (cohort 2: patients in the hospital) are treated. Patients receive two weeks of K+CS at a dose of 20 mg three times a day of KPT-330 and 1500 mg of CS three times a day for 14 days. Primary outcome for the cohort 1 is rate of hospital admission and the primary outcome for cohort 2 is days free from respiratory failure. Rate of viral clearance, coagulopathy, antibody formation and economic analysis are also assessed as secondary outcomes.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal having cancer, wherein said method comprises administering (a) an inhibitor of a chromosomal maintenance 1 (CRM1) polypeptide and (b) a salicylate to said mammal to reduce the number of cancer cells in said mammal.

2. The method of claim 1, wherein said mammal is a human.

3. (canceled)

4. The method of claim 1, wherein said cancer is selected from the group consisting of a diffuse large B-cell lymphoma (DLBCL), a T-cell lymphoma (TCL), a mantle cell lymphoma (MCL), a non-Hodgkin lymphoma (NHL), multiple myeloma (MM), Hodgkin lymphoma, small lymphocytic lymphoma, lymphoplasmacytic lymphoma, chronic lymphocytic leukemia, chronic lymphocytic leukemia, acute myelogenous leukemia, chronic myelogenous leukemia, myeloproliferative syndromes, and myelodysplastic syndromes.

5. The method of claim 1, wherein said inhibitor of a CRM1 polypeptide is selected from the group consisting of selinexor, leptomycin B, KPT-185, KPT-276, eltanexor, piperlongumine, verdinexor, valtrate, and ratjadone C.

6. (canceled)

7. The method of claim 1, wherein said salicylate is selected from the group consisting of aspirin, choline salicylate, sodium salicylate, acetyl salicylate, and choline magnesium trisalicylate.

8. The method of claim 1, wherein said inhibitor of a CRM1 polypeptide results in a plasma concentration within said mammal of from about 0.01 nM to about 1.25 μM, and wherein said salicylate results in a plasma concentration within said mammal of from about 0.1 μM to about 10 mM.

9. A method for treating a mammal having cancer, wherein said method comprises administering (a) an inhibitor of a CRM1 polypeptide and (b) a salicylate to said mammal to arrest the cell cycle of a cancer cell in said mammal.

10. The method of claim 9, wherein said cell cycle is arrested at a S phase.

11. The method of claim 9, wherein said mammal is a human.

12. (canceled)

13. The method of claim 9, wherein said cancer is selected from the group consisting of a diffuse large B-cell lymphoma (DLBCL), a T-cell lymphoma (TCL), a mantle cell lymphoma (MCL), a non-Hodgkin lymphoma (NHL), multiple myeloma (MM), Hodgkin lymphoma, small lymphocytic lymphoma, lymphoplasmacytic lymphoma, chronic lymphocytic leukemia, chronic lymphocytic leukemia, acute myelogenous leukemia, chronic myelogenous leukemia, myeloproliferative syndromes, and myelodysplastic syndromes.

14. The method of claim 9, wherein said inhibitor of a CRM1 polypeptide is CRM1 polypeptide is selected from the group consisting of selinexor, leptomycin B, KPT-185, KPT-276, eltanexor, piperlongumine, verdinexor, valtrate, and ratjadone C.

15. (canceled)

16. The method of claim 9, wherein said salicylate is selected from the group consisting of aspirin, choline salicylate, sodium salicylate, acetyl salicylate, and choline magnesium trisalicylate.

17. The method of claim 9, wherein said inhibitor of a CRM1 polypeptide results in a plasma concentration within said mammal of from about 0.01 nM to about 1.25 μM, and wherein said salicylate results in a plasma concentration within said mammal of from about 0.1 μM to about 10 mM.

18. A method for treating a mammal having a viral infection, wherein said method comprises administering (a) an inhibitor of a chromosomal maintenance 1 (CRM1) polypeptide and (b) a salicylate to said mammal to reduce the number of viral particles in said mammal.

19. The method of claim 1, wherein said mammal is a human.

20. The method of claim 18, wherein said viral infection is caused by a coronavirus.

21. The method of claim 20, wherein said coronavirus is a beta-coronavirus.

22. The method of claim 21, wherein said virus is SARS-CoV-2.

23. The method of claim 18, wherein said inhibitor of a CRM1 polypeptide is selected from the group consisting of selinexor, leptomycin B, KPT-185, KPT-276, eltanexor, piperlongumine, verdinexor, valtrate, and ratjadone C.

24. (canceled)

25. The method of claim 18, wherein said salicylate is selected from the group consisting of aspirin, choline salicylate, sodium salicylate, acetyl salicylate, and choline magnesium trisalicylate.
